(19) United States
(12) Patent Application Publication
(10) Pub. No.: US 2025/0213762 A1
(43) Pub. Date: Jul. 3, 2025

(54) APPARATUS AND METHODS FOR A DIALYSIS SYSTEM HAVING AN ULTRAFILTER

(71) Applicants: BAXTER INTERNATIONAL INC., Deerfield, IL (US); BAXTER HEALTHCARE SA, Glattpark (Opfikon) (CH)

(72) Inventors: Mauro GUSELLA, Castelvetro di Modena (IT); Mauro SUFFRITTI, Medolla Modena (IT); Oskar Erik Frode Styrbjorn FALLMAN, Lund (SE); Andrea DI BUONO, Carpi MO (IT); Ralf FLIEG, Rangendingen (DE); John Anders WELLINGS, Belleair Beach, FL (US); Ramkumar JEYACHANDRAN, Whitefield Bangalore (IN)

(21) Appl. No.: 19/003,794

(22) Filed: Dec. 27, 2024

(30) Foreign Application Priority Data
Dec. 28, 2023 (IN) 202341089537

(51) Int. Cl. A61M 1/16; A61M 1/28

(52) U.S. Cl. CPC A61M 1/1603; A61M 1/28; A61M 2205/3355; A61M 2205/705; A61M 2205/75; A61M 2210/1017

(57) ABSTRACT

A peritoneal dialysis (“PD”) system having an ultrafilter is disclosed herein. In one example, the PD system includes a housing and a PD fluid pump. The PD system also includes a filter comprising an outer chamber, a central portion, a membrane, an inlet connected to the outer chamber, an outlet, a first venting port connected to the outer chamber, and a second venting port connected to the central portion. The first venting port has a valve preventing air flow into the filter via the first venting port. The PD system further includes a pressure sensor and a control unit configured to control the PD fluid pump. The control unit is further configured to determine a pressure inside the filter based on an output from the pressure sensor and determine an integrity status of the membrane based on the pressure inside the filter.

### PRIORITY CLAIM

This application claims priority to and the benefit as a non-provisional application of Indian Provisional Patent Application No. 202341089537, filed Dec. 28, 2023, the entire contents of which are hereby incorporated by reference and relied upon.

### TECHNICAL FIELD

The present disclosure relates generally to medical fluid treatments, and in particular to dialysis fluid treatments that use fluid filtering.

### BACKGROUND

Due to various causes, a person's renal system can fail. Renal failure produces several physiological derangements. For instance, it is no longer possible to balance water and minerals or to excrete daily metabolic load. Additionally, toxic end products of metabolism, such as urea, creatinine, uric acid, and others, may accumulate in a patient's blood and tissue.

Reduced kidney function and, above all, kidney failure is treated with dialysis. Dialysis removes waste, toxins, and excess water from the body that normal functioning kidneys would otherwise remove. Dialysis treatment for the replacement of kidney functions is critical to many people because the treatment is lifesaving.

One type of kidney failure therapy is Hemodialysis (“HD”), which in general uses diffusion to remove waste products from a patient's blood. A diffusive gradient occurs across a semi-permeable dialyzer between the blood and an electrolyte solution, called dialysate or dialysis fluid, to cause diffusion.

Hemofiltration (“HF”) is an alternative renal replacement therapy that relies on a convective transport of toxins from a patient's blood. HF is accomplished by adding substitution or replacement fluid to an extracorporeal circuit during treatment. The substitution fluid and the fluid accumulated by the patient in between treatments is ultrafiltered over the course of the HF treatment, providing a convective transport mechanism that is particularly beneficial in removing middle and large molecules.

Hemodiafiltration (“HDF”) is a treatment modality that combines convective and diffusive clearances. HDF uses dialysis fluid flowing through a dialyzer, similar to standard hemodialysis, to provide diffusive clearance. In addition, substitution solution is provided directly to the extracorporeal circuit, providing convective clearance.

Most HD, HF, and HDF treatments occur in centers. A trend towards home hemodialysis (“HHD”) exists today in part because HHD can be performed daily, offering therapeutic benefits over in-center hemodialysis treatments, which occur typically bi- or tri-weekly. Studies have shown that more frequent treatments remove more toxins and waste products and render less interdialytic fluid overload than a patient receiving less frequent but perhaps longer treatments. A patient receiving more frequent treatments does not experience as much of a down cycle (swings in fluids and toxins) as does an in-center patient, who has built-up two or three days' worth of toxins prior to a treatment. In certain areas, the closest dialysis center can be many miles from the patient's home, causing door-to-door treatment time to consume a large portion of the day. Treatments in centers close to the patient's home may also consume a large portion of the patient's day. HHD can take place overnight or during the day while the patient relaxes, works or is otherwise productive.

Another type of kidney failure therapy is peritoneal dialysis (“PD”), which infuses a dialysis solution, also called dialysis fluid, into a patient's peritoneal chamber via a catheter. The dialysis fluid is in contact with the peritoneal membrane in the patient's peritoneal chamber. Waste, toxins, and excess water pass from the patient's bloodstream, through the capillaries in the peritoneal membrane, and into the dialysis fluid due to diffusion and osmosis, i.e., an osmotic gradient occurs across the membrane. An osmotic agent in the PD dialysis fluid provides the osmotic gradient. Used or spent dialysis fluid is drained from the patient, removing waste, toxins, and excess water from the patient. This cycle is repeated, e.g., multiple times.

There are various types of peritoneal dialysis therapies, including continuous ambulatory peritoneal dialysis (“CAPD”), automated peritoneal dialysis (“APD”), tidal flow dialysis, and continuous flow peritoneal dialysis (“CFPD”). CAPD is a manual dialysis treatment. Here, the patient manually connects an implanted catheter to a drain to allow used or spent dialysis fluid to drain from the peritoneal chamber. The patient then switches fluid communication so that the patient catheter communicates with a bag of fresh dialysis fluid to infuse the fresh dialysis fluid through the catheter and into the patient. The patient disconnects the catheter from the fresh dialysis fluid bag and allows the dialysis fluid to dwell within the peritoneal chamber, where the transfer of waste, toxins, and excess water takes place. After a dwell period, the patient repeats the manual dialysis procedure, for example, four times per day. Manual peritoneal dialysis requires a significant amount of time and effort from the patient, leaving ample room for improvement.

Automated peritoneal dialysis (“APD”) is similar to CAPD in that the dialysis treatment includes drain, fill, and dwell cycles. Automated PD machines, however, perform the cycles automatically, typically while the patient sleeps. The PD machines free patients from having to manually perform the treatment cycles and from having to transport supplies during the day. The PD machines connect fluidly to an implanted catheter, to a source or bag of fresh dialysis fluid and to a fluid drain. The PD machines pump fresh dialysis fluid from a dialysis fluid source, through the catheter and into the patient's peritoneal chamber. The PD machines also allow for the dialysis fluid to dwell within the chamber and for the transfer of waste, toxins, and excess water to take place. The source may include multiple liters of dialysis fluid including several solution bags.

The PD machines pump used or spent dialysate from the patient's peritoneal cavity, though the catheter, to drain. As with the manual process, several drain, fill, and dwell cycles occur during dialysis. A “last fill” may occur at the end of an APD treatment. The last fill fluid may remain in the peritoneal chamber of the patient until the start of the next treatment or may be manually emptied at some point during the day.

It is desirable for the dialysis fluid delivered to the patient's peritoneal chamber to be free from impurities such as bacterial or endotoxin contaminants. As such, a method to reduce concentration of contaminants is accordingly needed.

### SUMMARY

The present disclosure sets forth an automated peritoneal dialysis (“PD”) system, which provides improved filtration of PD fluid. The system includes a PD machine or cycler. The PD machine is configured to deliver fresh, heated PD fluid to a patient at, for example, 14 kPa (2.0 psig) or lower. The PD machine is capable of removing used PD fluid or effluent from the patient at, for example, −9 kPa (−1.3 psig) or higher. Fresh PD fluid is delivered via a single or dual lumen patient line to the patient and is first heated to a body fluid temperature, e.g., 37° C. The heated PD fluid is pumped through an ultrafilter and then pumped through the patient line. In some examples, the PD fluid is additionally pumped through a disposable filter set. The disposable filter set communicates fluidly with the patient line. The disposable filter set includes one or more sterilizing grade filter membranes that further filter fresh PD fluid. The ultrafilter and the disposable filter set are provided in one embodiment as last chance filters for the PD machine, which has been heat disinfected between treatments. Any bacterial or endotoxin contaminants that may remain after disinfection, albeit unlikely, are filtered from the PD fluid via the ultrafilter and the sterilizing grade filter membrane(s) of the disposable filter. Alternatively, the ultrafilter may provide filtration of PD fluid, water, or cleansing fluid for cleansing of the PD machine. The ultrafilter is intended to remove bacteria, such as E. coli and P. aeruginosa, as well as endotoxins. The ultrafilter may be one such as those made by Medica SRL, Mirandola, Italy, by Nipro Corp., Osaka, Japan, or by Baxter Healthcare Corporation, Deerfield, Illinois, United States.

It is desirable to test the one or more filters of the PD system to ensure the filtering membranes of the filters maintain integrity and successfully exclude target molecules from the PD fluid. Thus, the PD system in one embodiment is configured to test the integrity of one or more membranes included in a filter. In some embodiments, the PD system is configured to test two or more filters that are included in the PD system. The PD system may test the integrity of the membranes by performing a five-phased method. In the first phase, the filter is primed by fresh fluid. In the second phase, air is removed from a drain line of the filter. In the third phase, integrity of a first membrane is tested by pumping fluid or air in to or out of the filter causing a pressure differential inside the filter on the first membrane and monitoring the pressure in the filter over a period of time. In an optional fourth phase, integrity of a second membrane is tested by pumping a second amount of fluid or air in to or out of the filter causing a pressure differential inside the filter on the first membrane and on the second membrane and monitoring the pressure over a second period of time. In the fifth phase, the pressure inside the filter is re-equalized. Using the five-phased method described above, a defect in the first or second membrane may be identified. The defect may include a hole, a broken fiber, or any other deformity which would affect the performance of the filter. In some examples, the five-phased method can identify a hole as small as 4-5 microns in diameter. In other examples, the minimum hole size identifiable by the five-phased method is larger or smaller than 4-5 microns.

The PD system in one embodiment is configured to perform an internal recirculation filtering process. During a disinfection progress, endotoxins may be generated in the solution, tubing (e.g., lines) or other components of the PD system. As such, it is desirable to remove the endotoxins from the PD system prior to a dialysis treatment. In known methods, fresh fluid is flushed through the PD system. For example fresh fluid is pumped into the PD system from a fluid source and through the lines and components of the PD system prior to exiting the PD system and being disposed down a drain. In order to achieve high logarithmic reduction values (LRV) of target molecules such as endotoxin units, a single pass of a large volume of fluid is performed. However, in some systems, such as home automated PD machines, an amount of fluid available to perform a flush is much more limited.

Therefore, the PD system in one embodiment disclosed herein is configured to recirculate a fixed flush volume of fluid through the PD system including one or more filters to increase LVR compared to a single pass with the same amount of flush volume. For example, the volume of fluid inside the PD system or a fixed volume of fluid is introduced from a fluid source and recirculated throughout the PD system on a recirculation circuit via a fluid pump. The fixed volume of fluid is pumped through the one or more filters of the PD system a number of times, each time increasing the concentration of excluded molecules on a concentrate side of the filter(s). The fixed volume of fluid is then flushed out of the PD system and down a drain by introduction of a second volume of fluid. In this manner, a high LRV can be obtained using significantly less flush volume than traditional flush methods.

In an alternative embodiment, the PD system does not include a disposable filter and instead includes an additional ultrafilter. In this embodiment, the two or more ultrafilters may need to be replaced periodically. In order to provide a sterile and user-friendly method for replacing the two or more ultrafilters, the two or more ultrafilters may be provided in an ultrafilter box. The ultrafilter box includes a single housing enclosing two or more fluidly connected ultrafilters. In this manner, a user (e.g., a patient) may be able to open a housing of the PD system, remove a used ultrafilter box, and install a new ultrafilter box.

In light of the disclosure set forth herein, and without limiting the disclosure in any way, in a first aspect of the present disclosure, which may be combined with any other aspect, or portion thereof, a peritoneal dialysis (“PD”) system includes a housing; a PD fluid pump housed by the housing; a filter including a first outer chamber, a central portion, a first membrane separating the first outer chamber and the central portion, an inlet connected to the first outer chamber, an outlet, a first venting port connected to the first outer chamber, the first venting port having a valve preventing air flow into the filter via the first venting port, and a second venting port connected to the central portion; a pressure sensor; and a control unit configured to control the PD fluid pump, the control unit further configured to determine a pressure inside the filter based on an output from the pressure sensor and determine an integrity status of the first membrane based on the pressure inside the filter.

In a second aspect of the present disclosure, which may be combined with any other aspect, or portion thereof, the filter is an ultrafilter, a membrane filter, or a sterile membrane filter.

In a third aspect of the present disclosure, which may be combined with any other aspect, or portion thereof, the filter is an ultrafilter, the first membrane includes a plurality of hollow fibers, the first outer chamber is a concentrate side of the filter, and the central portion is a filtrate side of the filter.

In a fourth aspect of the present disclosure, which may be combined with any other aspect, or portion thereof, the filter further includes a second outer chamber and a second membrane separating the central portion and the second outer chamber and the control unit is further configured to determine an integrity state of the second membrane based on the pressure inside the filter.

In a fifth aspect of the present disclosure, which may be combined with any other aspect, or portion thereof, the filter further includes at least one of a first supporting structure between the first outer chamber and the central portion and a second supporting structure between the central portion and the second outer chamber.

In a sixth aspect of the present disclosure, which may be combined with any other aspect, or portion thereof, the filter further includes a supply line connected to the inlet and a drain line connected to the outlet.

In a seventh aspect of the present disclosure, which may be combined with any other aspect, or portion thereof, the filter further includes a housing and wherein the first outer chamber and the central portion are within the housing.

In an eighth aspect of the present disclosure, which may be combined with any other aspect, or portion thereof, a peritoneal dialysis (“PD”) system includes a housing; a PD fluid pump housed by the housing; a filter set including a first filter having a first outer chamber and a first central chamber, a second filter having a second central chamber and a second outer chamber, a first membrane separating the first outer chamber and the first central chamber, a second membrane separating the second central chamber and the second outer chamber, an inlet connected to the first outer chamber, an outlet connected to the second outer chamber, a first venting port connected to the first outer chamber, the first venting port having a valve preventing air flow into the filter set via the first venting port, a second venting port connected to the first central chamber, and a third venting port connected to the second central chamber; a pressure sensor; and a control unit configured to control the PD fluid pump, the control unit further configured to determine a pressure inside the filter set based on an output from the pressure sensor and determine an integrity status of the first membrane and the second membrane based on the pressure inside the filter set.

In a ninth aspect of the present disclosure, which may be combined with any other aspect, or portion thereof, the first filter is one of an ultrafilter or a membrane filter and the second filter is one of an ultrafilter or a membrane filter.

In a tenth aspect of the present disclosure, which may be combined with any other aspect, or portion thereof, the filter set further includes a first housing and a second housing.

In an eleventh aspect of the present disclosure, which may be combined with any other aspect, or portion thereof, the first outer chamber and the first central chamber are within the first housing and the second central chamber and the second outer chamber are within the second housing.

In a twelfth aspect of the present disclosure, which may be combined with any other aspect, or portion thereof, the system further includes fluid connection between the first filter and the second filter.

In a thirteenth aspect of the present disclosure, which may be combined with any other aspect, or portion thereof, a peritoneal dialysis (“PD”) method for testing integrity of a filter includes causing a PD fluid pump, via a control unit of a PD machine, to fill the filter with fluid via an inlet of the filter; causing the PD fluid pump, via the control unit, to remove a portion of the fluid via a drain line until a pressure in the filter reaches a first pressure threshold; causing the PD fluid pump, via the control unit, to refill the filter with fluid via the inlet of the filter until the pressure in the filter is substantially equalized, wherein refilling the filter with fluid causes a portion of a drain line of the filter proximate to a cap to fill with fluid; causing the PD fluid pump, via the control unit, to remove a second portion of the fluid via the inlet of the filter until a pressure in a first chamber of the filter reaches a second pressure threshold; monitoring, via a pressure sensor controlled by the control unit, the pressure in the first chamber for a first period of time; and determining, via the control unit, an integrity status of a first membrane of the filter based on the pressure in the first chamber during the first period of time.

In a fourteenth aspect of the present disclosure, which may be combined with any other aspect, or portion thereof, the PD method further comprises causing the filter, via the control unit, to equalize the pressure in the first chamber after monitoring the pressure in the first chamber.

In a fifteenth aspect of the present disclosure, which may be combined with any other aspect, or portion thereof, the PD method further comprises determining, via the control unit, the first membrane does not include at least one hole or deformity in response to the pressure in the first chamber remaining stable for the first period of time.

In a sixteenth aspect of the present disclosure, which may be combined with any other aspect, or portion thereof, the PD method further comprises determining, via the control unit, the first membrane includes one or more holes or deformities in response to the pressure in the first chamber increasing or decreasing during the first period of time.

In a seventeenth aspect of the present disclosure, which may be combined with any other aspect, or portion thereof, the PD method further comprises causing the PD fluid pump, via the control unit, to remove a third portion of the fluid via the drain line until a pressure in a second chamber of the filter and a pressure in the first chamber of the filter reach a third pressure threshold; monitoring, via the pressure sensor controlled by the control unit, the pressure in the first chamber and the second chamber for a second period of time; and determining, via the control unit, an integrity status of a second membrane of the filter based on the pressure in the first chamber and the second chamber during the second period of time.

In an eighteenth aspect of the present disclosure, which may be combined with any other aspect, or portion thereof, the PD method further comprises communicating, via the control unit, the integrity status of the first membrane and the integrity status of the second membrane.

In a nineteenth aspect of the present disclosure, which may be combined with any other aspect, or portion thereof, the PD method further comprises determining, via the control unit, the second membrane does not include a at least one hole or deformity in response to the pressure in the first chamber and the second chamber remaining stable for the second period of time.

In a twentieth aspect of the present disclosure, which may be combined with any other aspect, or portion thereof, the PD method further comprises determining, via the control unit, the second membrane includes one or more holes or deformities in response to the pressure in the first chamber and the second chamber increasing or decreasing during the second period of time.

In a twenty-first aspect of the present disclosure, which may be combined with any other aspect, or portion thereof, a peritoneal dialysis (“PD”) method for removing contaminants includes opening, via a control unit of a PD machine, a first valve connected to a first outlet of a filter; closing, via the control unit, a second valve connected to a second outlet of the filter and a third valve connected to a third outlet of a filter; controlling, via the control unit, a pump to push a first volume of fluid from a concentrate side to a filtrate side of the filter, wherein the fluid flows from an inlet of the filter to the first outlet of the filter, passing through one or more membranes of the filter, wherein the fluid flows through a recirculation circuit back to the inlet of the filter after flowing out the first outlet of the filter; closing, via the control unit, the first valve of the filter; opening, via the control unit, the third valve of the filter and a fourth valve, the fourth valve providing fluid connection between a fluid source and the recirculation circuit; and controlling, via the control unit, the pump to push a second volume of fluid from the fluid source and the first volume of fluid to a drain, wherein the first volume and the second volume flow from the inlet of the filter to the third outlet of the filter before flowing to the drain.

In a twenty-second aspect of the present disclosure, which may be combined with any other aspect, or portion thereof, the PD method further includes prior to the opening of the third valve and the fourth valve, closing, via the control unit, the first valve of the filter; and opening, via the control unit, the second valve of the filter, wherein the fluid flows from the inlet of the filter to the second outlet of the filter before flowing through the recirculation circuit.

In a twenty-third aspect of the present disclosure, which may be combined with any other aspect, or portion thereof, the PD method further includes closing, via the control unit, the third valve and the fourth valve; opening, via the control unit, the first valve; and controlling, via the control unit, the pump to push a third volume of fluid through the recirculation circuit.

In a twenty-fourth aspect of the present disclosure, which may be combined with any other aspect, or portion thereof, a peritoneal dialysis (“PD”) system includes a housing; a PD fluid pump housed by the housing; a filter; a fluid source; and a control unit configured to control the PD fluid pump, the control unit further configured to: cause the PD fluid pump to pump a volume of fluid through a recirculation circuit including the filter such that the volume of fluid passes through the filter two or more times.

In a twenty-fifth aspect of the present disclosure, which may be combined with any other aspect, or portion thereof, the control unit is further configured to cause the PD fluid pump to pump a second volume of fluid from the fluid source through the filter and cause the first volume and the second volume to exit the PD system at a drain.

In a twenty-sixth aspect of the present disclosure, which may be combined with any other aspect, or portion thereof, the filter is an ultrafilter.

In a twenty-seventh aspect of the present disclosure, which may be combined with any other aspect, or portion thereof, the filter includes a first outlet and a second outlet in fluid communication with a filtrate portion of the filter; and a third outlet in fluid communication with a concentrate portion of the filter.

In a twenty-eighth aspect of the present disclosure, which may be combined with any other aspect, or portion thereof, the recirculation circuit is in fluid connection with one or more of the first outlet and the second outlet; and the third outlet is in fluid communication with a drain.

In a twenty-ninth aspect of the present disclosure, which may be combined with any other aspect, or portion thereof, an inlet of the filter is in fluid communication with the concentrate portion of the filter.

In a thirtieth aspect of the present disclosure, which may be combined with any other aspect, or portion thereof, the PD system further includes a first valve on a first fluid line in fluid communication with of the first outlet, the first valve controlling fluid flow out of the first outlet; a second valve on a second fluid line in fluid communication with the second outlet, the second valve controlling fluid flow out of the second outlet; a third valve on a third fluid line in fluid communication with the third outlet, the third valve controlling fluid flow out of the third outlet; and a fourth valve on a fourth fluid line in fluid communication with the fluid source, the fourth valve controlling fluid flow out of the fluid source.

In a thirty-first aspect of the present disclosure, which may be combined with any other aspect, or portion thereof, the control unit is further configured to control one or more of the first valve, the second valve, the third valve, and the fourth valve.

In a thirty-second aspect of the present disclosure, which may be combined with any other aspect, or portion thereof, the control unit is further configured to cause one or more of the first valve and the second valve to open, the third valve and the fourth valve to close, and the PD fluid pump to operate when the PD system is in a recirculation mode.

In a thirty-third aspect of the present disclosure, which may be combined with any other aspect, or portion thereof, the control unit is further configured to cause the first valve and the second valve to close, the third valve and the fourth valve to open, and the PD fluid pump to operate when the PD system is in a flush mode.

In a thirty-fourth aspect of the present disclosure, which may be combined with any other aspect, or portion thereof, a peritoneal dialysis (“PD”) system includes a housing; a PD fluid pump housed by the housing; a fluid source; a control unit configured to control the PD fluid pump; and an ultrafilter box having a box housing, the box housing enclosing a first ultrafilter having a first ultrafilter housing and a second ultrafilter having a second ultrafilter housing, the ultrafilter box contained within the housing of the PD system.

In a thirty-fifth aspect of the present disclosure, which may be combined with any other aspect, or portion thereof, any of the features, functionality and alternatives described in connection with any one or more of FIGS. 1 to 25 may be combined with any of the features, functionality and alternatives described in connection with any other of FIGS. 1 to 25.

In light of the above aspects and present disclosure set forth herein, it is an advantage of the present disclosure to provide a system and method for testing the integrity of one or more membranes of one or more ultrafilters.

It is another advantage of the present disclosure to provide a PD fluid system that flushes contaminants from the system using a minimal amount of fluid.

It is a further advantage of the present disclosure to provide a PD fluid system that provides high logarithmic reduction value (LRV) of contaminants during flush of the system.

Moreover, it is an advantage of the present disclosure to provide a PD fluid system that provides a user-friendly configuration for set-up and replacement of multiple ultrafilters.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a fluid flow schematic of one embodiment for a medical fluid, e.g., PD fluid, system that is set for treatment.

FIG. 2A is a front view of a PD machine according to the present disclosure.

FIG. 2B is a second front view of a PD machine according to the present disclosure with the PD machine in an open configuration.

FIG. 3 is a fluid flow schematic of one embodiment of the medical fluid system of the present disclosure showing emptying a first pathway.

FIG. 4 is a fluid flow schematic of one embodiment of the medical fluid system of the present disclosure showing emptying a second pathway.

FIG. 5 is a fluid flow schematic of one embodiment of the medical fluid system of the present disclosure showing a emptying a third pathway.

FIG. 6 is a fluid flow schematic of one embodiment of the medical fluid system of the present disclosure showing fluid flow for an integrity test.

FIG. 7 is a fluid flow schematic of one embodiment of the medical fluid system of the present disclosure showing fluid flow for flushing the solution lines.

FIG. 8 is a fluid flow schematic of one embodiment of the medical fluid system of the present disclosure showing a second example of fluid flow for flushing the solution lines.

FIG. 9 is a fluid flow schematic of one embodiment of the medical fluid system of the present disclosure showing a third example of fluid flow for flushing the solution lines.

FIG. 10 is a fluid flow schematic of one embodiment of the medical fluid system of the present disclosure showing a fourth example of fluid flow for flushing the solution lines.

FIG. 11 is a fluid flow schematic of one embodiment of the medical fluid system of the present disclosure showing an example of fluid flow for priming the air trap.

FIG. 12 is a fluid flow schematic of one embodiment of the medical fluid system of the present disclosure showing an example of fluid flow for priming the ultrafilter(s).

FIG. 13 is a fluid flow schematic of one embodiment of the medical fluid system of the present disclosure showing an example of fluid flow after a completed flush.

FIG. 14 is a fluid flow schematic of one embodiment of the medical fluid system of the present disclosure showing an example of fluid flow during fill.

FIG. 15 is a fluid flow schematic of one embodiment of the medical fluid system of the present disclosure showing an example of fluid flow during drain.

FIG. 16 is a fluid flow schematic of one embodiment of the medical fluid system of the present disclosure showing an example of fluid flow during filling of the air trap.

FIG. 17 is a fluid flow schematic of one embodiment of the medical fluid system of the present disclosure showing an example of fluid flow during tear-down.

FIG. 18 is a fluid flow schematic of one embodiment of the medical fluid system of the present disclosure showing a second example of fluid flow during tear-down.

FIG. 19 is a fluid flow schematic of one embodiment of the medical fluid system of the present disclosure showing an example of fluid flow for pre-disinfection tear-down.

FIG. 20 is a fluid flow schematic of one embodiment of the medical fluid system of the present disclosure showing an example of fluid flow during disinfection.

FIG. 21 is a fluid flow schematic of one embodiment of the medical fluid system of the present disclosure showing a second example of fluid flow during disinfection.

FIG. 22 is a fluid flow schematic of one embodiment of the medical fluid system of the present disclosure showing a third example of fluid flow during disinfection.

FIG. 23A is a schematic of one embodiment of a filter of the medical fluid system of the present disclosure showing a filter integrity test set-up.

FIG. 23B is a schematic of one embodiment of the filter of the medical fluid system of the present disclosure showing a first phase of the filter integrity test.

FIG. 23C is a schematic of one embodiment of the filter of the medical fluid system of the present disclosure showing a second phase of the filter integrity test.

FIG. 23D is a schematic of one embodiment of the filter of the medical fluid system of the present disclosure showing a second portion of the second phase of the filter integrity test.

FIG. 23E is a schematic of one embodiment of the filter of the medical fluid system of the present disclosure showing a third phase of the filter integrity test.

FIG. 23F is a schematic of one embodiment of the filter of the medical fluid system of the present disclosure showing a fourth phase of the filter integrity test.

FIG. 23G is a schematic of one embodiment of the filter of the medical fluid system of the present disclosure showing a fifth phase of the filter integrity test.

FIG. 23H is a schematic of another embodiment of the filter of the medical fluid system of the present disclosure showing a filter integrity test set-up.

FIG. 24A is a schematic of one embodiment of the medical fluid system of the present disclosure showing a recirculation circuit.

FIG. 24B is a schematic of one embodiment of the medical fluid system of the present disclosure showing a flush configuration.

FIG. 25 is a schematic of one embodiment of the medical fluid system of the present disclosure showing a multiple ultrafilter configuration.

### DETAILED DESCRIPTION

### System Overview

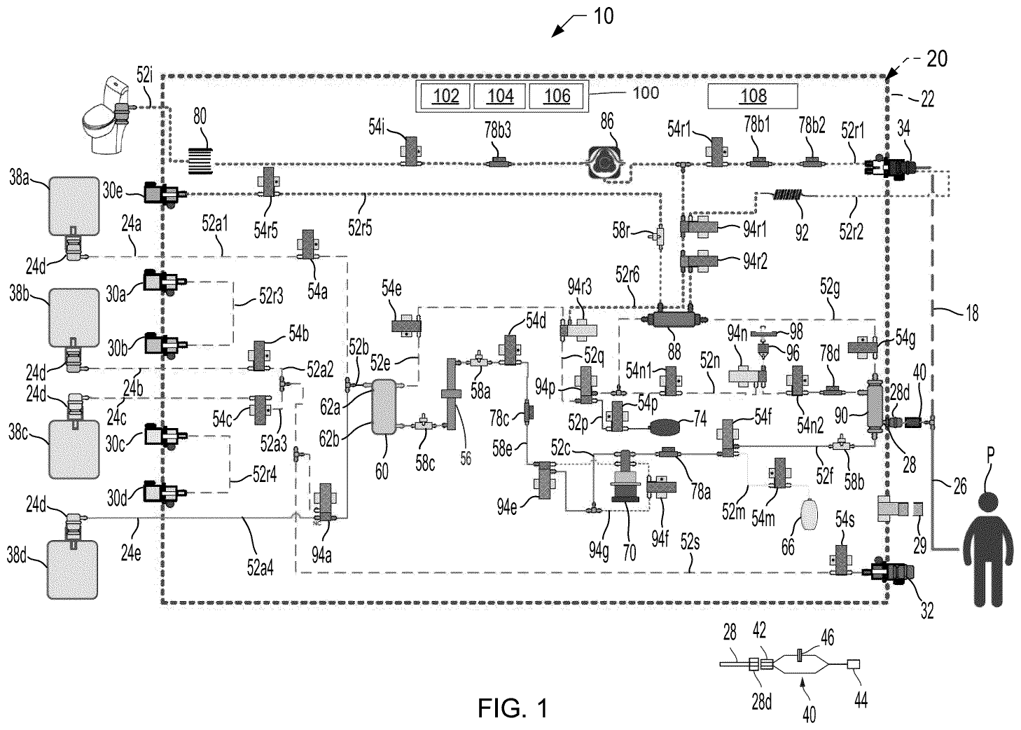

Referring now to the drawings and in particular to FIG. 1, a medical system having the ultrafilter of the present disclosure is illustrated via a peritoneal dialysis (“PD”) system 10. The example system 10 includes a PD machine or cycler 20 and a control unit 100 having one or more processor 102, one or more memory 104, video controller 106, and user interface 108. The example user interface 108 may alternatively or additionally be a remote user interface, e.g., via a tablet or smartphone. The control unit 100 may also include a transceiver and a wired or wireless connection to a network (not illustrated), e.g., the internet, for sending treatment data to and receiving prescription instructions/changes from a doctor's or clinician's server interfacing with a doctor's or clinician's computer. The control unit 100 in an embodiment controls all electrical, fluid flow, and heating components of the system 10 and receives outputs from sensors of the system 10. System 10 in the illustrated embodiment includes durable and reusable components that contact fresh and used PD fluid, which necessitates that the PD machine or cycler 20 be disinfected between treatments, e.g., via heat disinfection and/or chemical disinfection.

System 10 in FIG. 1 includes an inline resistive heater 56, reusable supply lines or tubes 52a1 to 52a4 and 52b, air trap 60 operating with respective upper and lower level sensors 62a and 62b, air trap valve 54d, vent valve 54e located along vent line 52e, reusable line or tubing 52c, PD fluid pump 70, temperature sensors 58a, 58b, 58c and 58r, pressure sensors 78a, 78b1, 78b2, 78b3, 78c and 78d, reusable patient tubing or lines 52f and 52g having respective valves 54f and 54g, reusable patient line 28, a hose reel 80 for retracting drain tubing or line 52i, and reusable drain tubing or line 18 extending to drain line connector 34.

Drain line connector 34 further connects to reusable line or tubing 52r1 having valve 54r1 and reusable line 52r2 having rigid silicone pipe 92. Reusable line 52r1 further connects to reusable line 52i having drain line valve 5412. Reusable line 52i further includes peristaltic pump 86. A third recirculation or disinfection tubing or line 52r3 extends between disinfection connectors 30a and 30b for use during disinfection. A fourth recirculation or disinfection tubing or line 52r4 extends between disinfection connectors 30c and 30d for use during disinfection. A fifth recirculation or disinfection tubing or line 52r5 having valve 54r5 extends between heat exchanger 88 and disinfection connector 30e. For disinfection, drain line 52i is in one embodiment connected to disinfection connector 30e to form a closed disinfection loop. Three-way valve 94r1 provides fluid connection between either reusable line or tubing 52r1 or reusable line or tubing 52r2 and the inlet of three-way valve 94r2. The three-way valve 94r2 provides fluid connection between the outlet of three-way valve 94r1 and either reusable line or tubing 52r6 or the heat exchanger 88. Three-way valve 94r3 provides fluid connection between either reusable line or tubing 52r6 or vent line 52e and reusable line or tubing 52q.

System 10 further includes PD fluid containers or bags 38a to 38c (e.g., holding the same or different formulations of PD fluid), which connect to distal ends 24d of reusable PD fluid lines 24a to 24c, respectively. System 10 further includes a fourth PD fluid container or bag 38d that connects to a distal end 24d of reusable PD fluid line 24e. Fourth PD fluid container or bag 38d may hold the same or different type (e.g., icodextrin) of PD fluid than provided in PD fluid containers or bags 38a to 38c. Reusable PD fluid lines 24a to 24c and 24e extend in one embodiment through apertures (not illustrated) defined or provided by housing 22 of the cycler 20. The illustrated embodiment of FIG. 1 also shows an external clamp 29 for the patient's transfer set 26.

System 10 in the illustrated embodiment includes four disinfection connectors 30a to 30d for connecting to distal ends 24d of reusable PD fluid lines 24a to 24c and 24e, respectively, during disinfection. Reusable supply tubing or lines 52a1 to 52a4 communicate with reusable supply lines 24a to 24c and 24e, respectively. Reusable supply tubing or lines 52a1 to 52a3 operate with valves 54a to 54c, respectively, to allow PD fluid from a desired PD fluid container or bag 38a to 38c to be pulled into cycler 20. Three-way valve 94a in the illustrated example allows for control unit 100 to select between (i) container or bag 38b or 38c and (ii) container or bag 38d. In the illustrated embodiment, fluid from container or bag 38d is connected to the normally closed port of three-way valve 94a.

System 10 also provides a connector 32 which, during flush or disinfection, directs air or fluid from ultrafilter 90 to reusable supply lines 52a1 to 52a4. Connector 32 connects to the reusable supply lines 52a1 to 52a4 via reusable line or tubing 52s having valve 54s.

PD fluid pump 70 may be an inherently accurate pump, such as a peristaltic pump, a piston pump, or less accurate pump, such as a gear pump that operates in cooperation with a flowmeter (not illustrated) to control fresh and used PD fluid flowrate and volume. The example system 10 further includes three-way valve 94e which provides fluid communication between reusable lines 58e and 52c. Three-way valve 94e may also provide fluid communication between reusable line 58e and reusable line 94g having valve 94f. In some embodiments, the three-way valve 94e enables PD fluid pump 70 to be bypassed during a test of an ultrafilter 90 and/or during disinfection.

In the illustrated example, system 10 is provided with an additional pressure sensor 78c located upstream of PD fluid pump 70, which allows for the measurement of the suction pressure of pump 70 to help control unit 100 more accurately determine pump volume. Additional pressure sensor 78c in the illustrated embodiment is located along reusable line 58e, which may be filled with air or a mixture of air and PD fluid, but which should nevertheless be at the same negative pressure as PD fluid located within PD fluid line 52c.

System 10 in the example of FIG. 1 includes reusable tubing 52n including three-way valve 94n. One port of three-way valve 94n is configured to connected to an air supply (not illustrated) via check valve 96 and sterile filter 98. Valves 54n1 and 54n2 are located along reusable line 52n.

System 10 in the example of FIG. 1 includes redundant pressure sensors 78b1 and 78b2, the output of one of which is used for pump control, as discussed herein, while the output of the other pressure sensor is a safety or watchdog output to make sure the control pressure sensor is operating accurately. Pressure sensors 78b1 and 78b2 are located along a line including recirculation valve 54r2.

System 10 in the example of FIG. 1 further includes a source of acid, such as a citric acid container or bag 66. Citric acid container or bag 66 is in selective fluid communication with three-way valve 54f via a citric acid valve 54m located along a citric acid line 52m. Citric acid line 52m is connected in one embodiment to the normally closed port of second three-way valve 54f, so as to provide redundant valves between citric acid container or bag 66 and the PD fluid circuit during treatment. The redundant valves ensure that no citric (or other) acid reaches the treatment fluid lines during treatment. Citric (or other) acid is instead used during non-therapy phases such as cleaning and/or disinfection.

System 10 in the example of FIG. 1 further includes a source of cleaner, such as a cleaner container or bag 74. Cleaner container or bag 74 is in selective fluid communication with three-way valve 94p via a cleaner valve 54p located along a cleaner line 52p. Cleaner line 52p is connected in one embodiment to the normally closed port of three-way valve 94p, so as to provide redundant valves between cleaner container or bag 74 and the PD fluid circuit during treatment. The redundant valves ensure that no cleaner reaches the treatment fluid lines during treatment. Cleaner is instead used during non-therapy phases such as cleaning and/or disinfection.

Control unit 100 in an embodiment uses feedback or an output from any one or more of pressure sensors 78a to 78d to enable PD machine 20 to deliver fresh, heated PD fluid to the patient at, for example, 14 kPa (2.0 psig) or higher. The pressure feedback is used to enable PD machine 20 to remove used PD fluid or effluent from the patient at, for example, −9 kPa (−1.3 psig) or higher. The pressure feedback may be used in a proportional, integral, derivative (“PID”) pressure routine for pumping fresh and used PD fluid at a desired positive or negative pressure.

Inline resistive heater 56 under control of control unit 100 is capable of heating fresh PD fluid to body temperature, e.g., 37° C., for delivery to patient P at a desired flowrate. Control unit 100 in an embodiment uses feedback from temperature sensor 58a in a PID temperature routine for pumping fresh PD fluid to patient P at a desired temperature.

System 10 in the example of FIG. 1 further includes an ultrafilter 90. The ultrafilter 90 is connected to reusable tubing 52f, 52g, and 52n as well as patient line 28. FIG. 1 also illustrates that system 10 includes and uses a disposable filter set 40, which communicates fluidly with the patient line 28, the patient's transfer set 26, and the drain tubing 52i. Disposable filter set 40 includes a disposable connector 42 that connects to a distal end 28e of reusable patient line 28. Disposable filter set 40 also includes a connector 44 that connects to the patient's transfer set. Disposable filter set 40 further includes a sterilizing grade filter membrane 46 that further filters fresh PD fluid. Disposable filter set 40 is provided in one embodiment as a last chance filter for PD machine 20, which has been heat disinfected between treatments. Any pathogens that may remain after disinfection, albeit unlikely, are filtered from the PD fluid via the sterilizing grade filter membrane 46 of disposable filter set 40. In some embodiments, the sterilizing grade filter membrane 46 may be replaced with a second ultrafilter that is fluidly coupled in series with the ultrafilter 90.

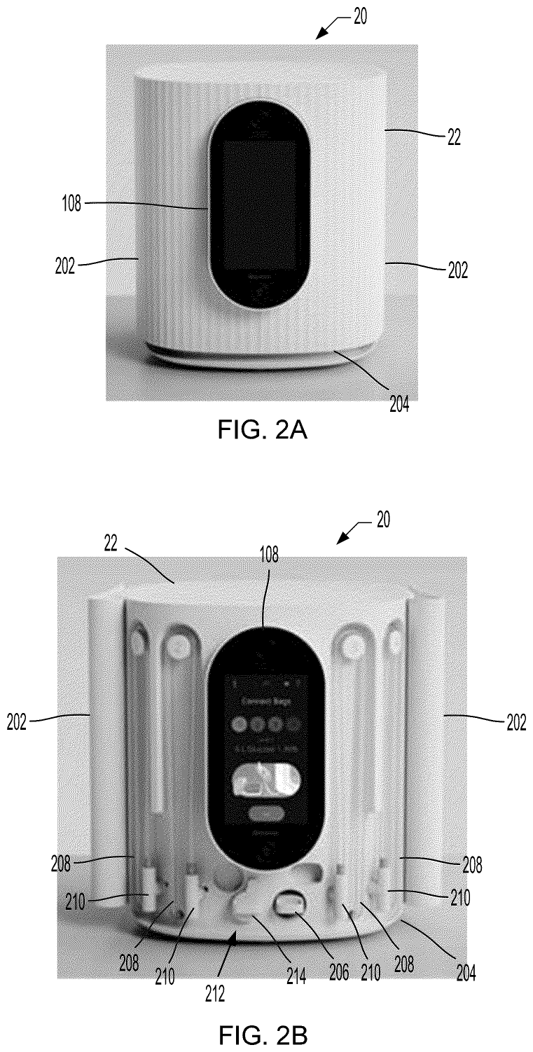

In FIG. 2A, a front view of an example embodiment of the PD machine 20 is illustrated. In the example of FIG. 2A, the PD machine 20 is illustrated in a closed configuration. The PD machine 20 may be placed in the closed configuration between dialysis treatments, during storage or transport, or during any other period of time for which the PD machine 20 is not in use. The PD machine 20 may also be placed in the closed configuration during disinfection of the PD machine 20. The example PD machine 20 of FIG. 2A includes the housing 22. The example housing 22 includes a plurality of housing doors 202. In the example of FIG. 2A, the housing 22 illustrated in a closed configuration such that the housing doors 202 cover a front portion 204 of the housing 22. The example PD machine 20 includes the user interface 108. In the closed configuration of the example housing 22, the example user interface 108 is not covered by the housing doors 202. The example user interface 108 of FIG. 2A is illustrated in a non-operational state such that no information is displayed on the user interface 108.

FIG. 2B illustrates a second front view of the PD machine 20. In the example of FIG. 2B, the PD machine 20 is illustrated in an open configuration. The PD machine 20 may be placed in the open configuration during a dialysis treatment, during set-up or take down of a dialysis treatment, during maintenance of the PD machine 20, or during any other period of time when the PD machine 20 is in use. Additionally, the PD machine 20 may be kept in the open configuration when the PD machine 20 is not in use. In the open configuration, the front portion 204 of the housing 22 is not covered by the housing doors 202. For example, the housing doors 202 are configured such that various components of the PD machine 20 are accessible by a user.

For example, in the open configuration, a user can access the patient line 206 (e.g., patient line 28 of FIG. 1) of the example PD machine 20. Example reusable tubing lines 208 (e.g., reusable supply lines 24a, 24b, 24c, 24e of FIG. 1) and reusable internal connectors 210 (e.g., disinfection connectors 30a to 30d of FIG. 1) are also accessible to a user in the open configuration of the PD machine 20. Additionally, the example PD machine 20 includes a drain line connection 212 (e.g., drain line connector 34 of FIG. 1) which is accessible in the open configuration of the PD machine 20. In the example of FIG. 2B, the drain line connection 212 is covered by a drain line connection cap 214. The example user interface 108 of FIG. 2B is illustrated in an operational state such that information about the PD machine 20 and/or instructions for a user are displayed. The example user interface 108 may further accept input from a user via a touch-screen interface and/or one or more buttons.

### Pre-Therapy Flush

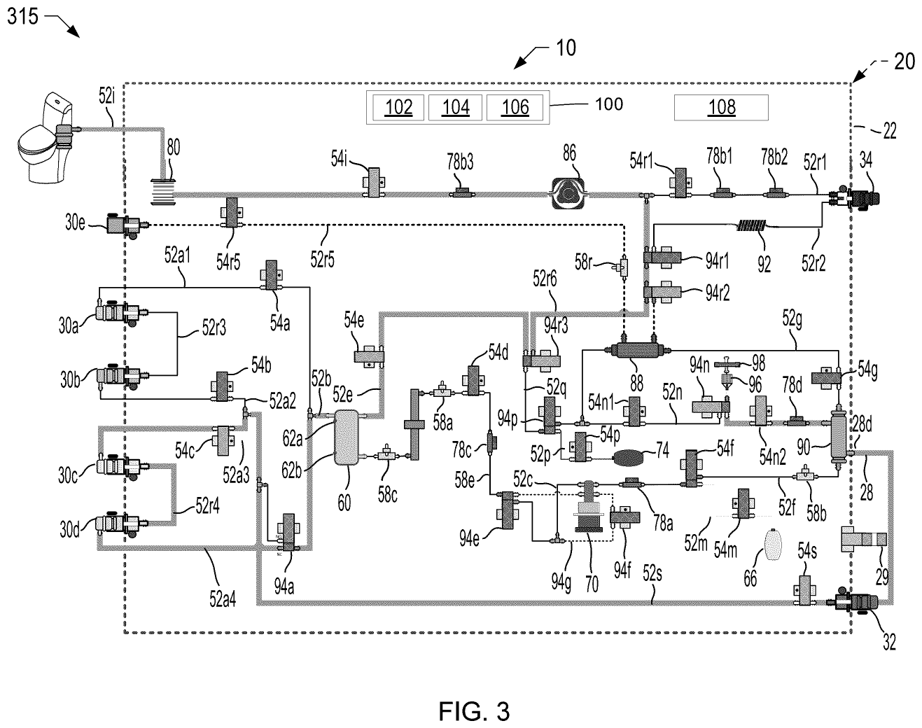

FIG. 3 illustrates a first fluid flow schematic 315 of one embodiment of the PD system 10. The first fluid flow schematic 315 illustrates an emptying a first pathway. For example, air is introduced into the system 10 via the air supply connected to the sterile filter 98. The sterile air travels through reusable tubing 52n, patient line 28, reusable tubing 52s, reusable supply line 52a3, recirculation tubing 52r4, reusable supply line 52a4, reusable supply line 52b, vent line 52e, reusable tubing 52r6, and drain tubing 52i. In some examples, the sterile air is introduced from a compressed air source such that the air pressure of the sterile air can empty the pathway of any fluid or solid particles.

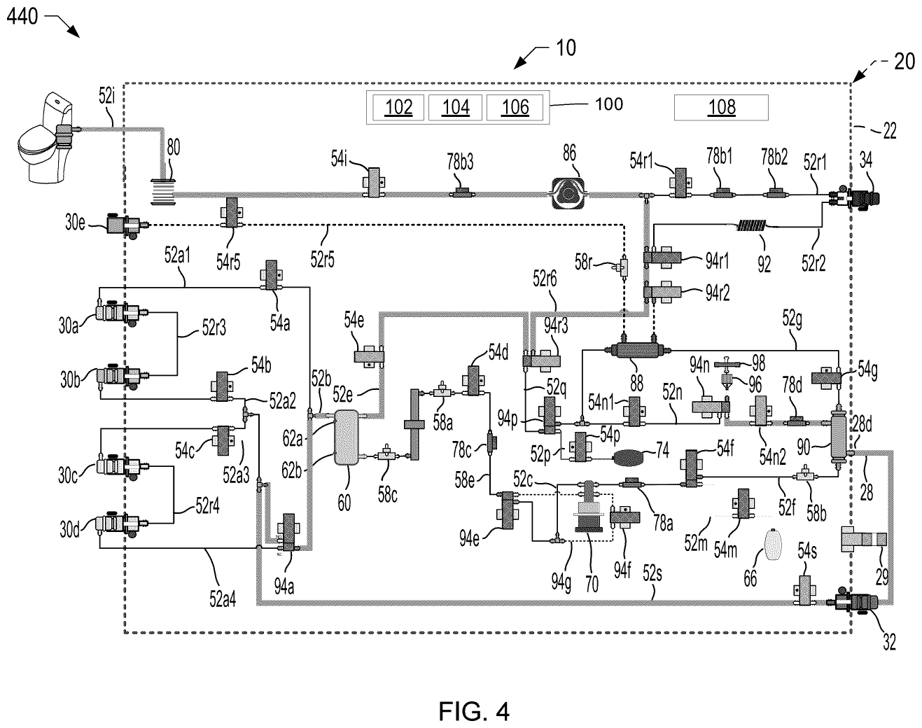

FIG. 4 illustrates a second fluid flow schematic 440 of one embodiment of the PD system 10. The second fluid flow schematic 440 illustrates emptying a second pathway. For example, air is introduced into the system 10 via the air supply connected to the sterile filter 98. The sterile air travels through reusable tubing 52n, patient line 28, reusable tubing 52s, reusable supply line 52a4, reusable supply line 52b, vent line 52e, reusable tubing 52r6, and drain tubing 52i. In some examples, the sterile air is introduced from a compressed air source such that the air pressure of the sterile air can empty the pathway of any fluid or solid particles. Emptying of the second pathway may occur immediately following emptying of the first pathway (e.g., by continuing to introduce sterile air while closing the valve 54c and valve 94a).

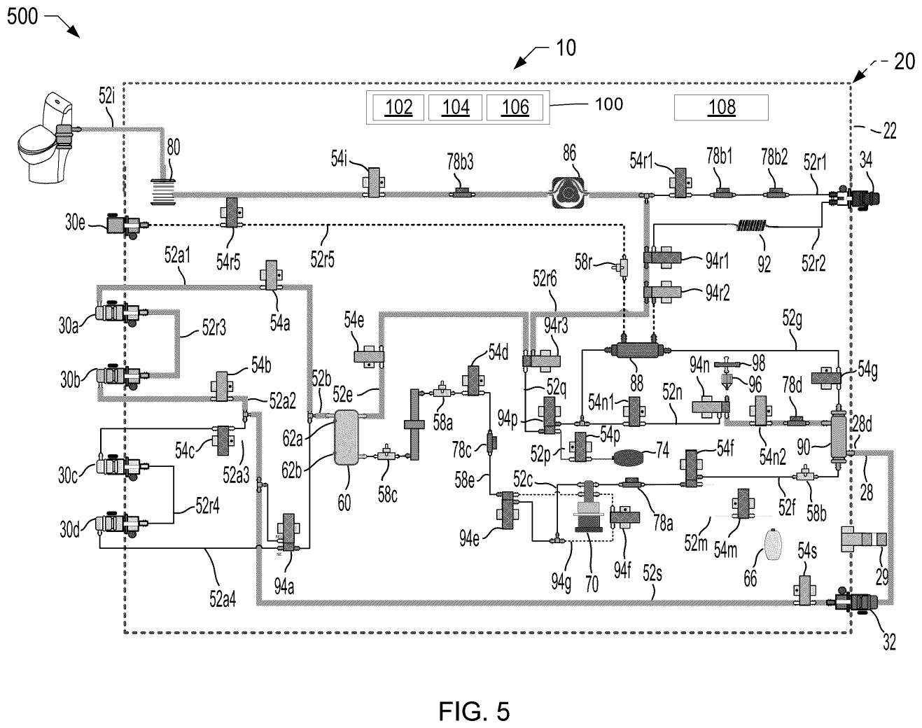

FIG. 5 illustrates a third fluid flow schematic 500 of one embodiment of the PD system 10. The third fluid flow schematic 500 illustrates emptying a third pathway. For example, air is introduced into the system 10 via the air supply connected to the sterile filter 98. The sterile air travels through reusable tubing 52n, patient line 28, reusable tubing 52s, reusable supply line 52a2, recirculation tubing 52r3, reusable supply line 52a1, reusable supply line 52b, vent line 52e, reusable tubing 52r6, and drain tubing 52i. In some examples, the sterile air is introduced from a compressed air source such that the air pressure of the sterile air can empty the pathway of any fluid or solid particles. Emptying of the third pathway may occur immediately following emptying of the first pathway (e.g., by continuing to introduce sterile air while closing the valve 54c and opening the valves 54b and 54a) or following emptying of the second pathway (e.g., by continuing to introduce sterile air while opening the valves 94a, 54b, and 54a).

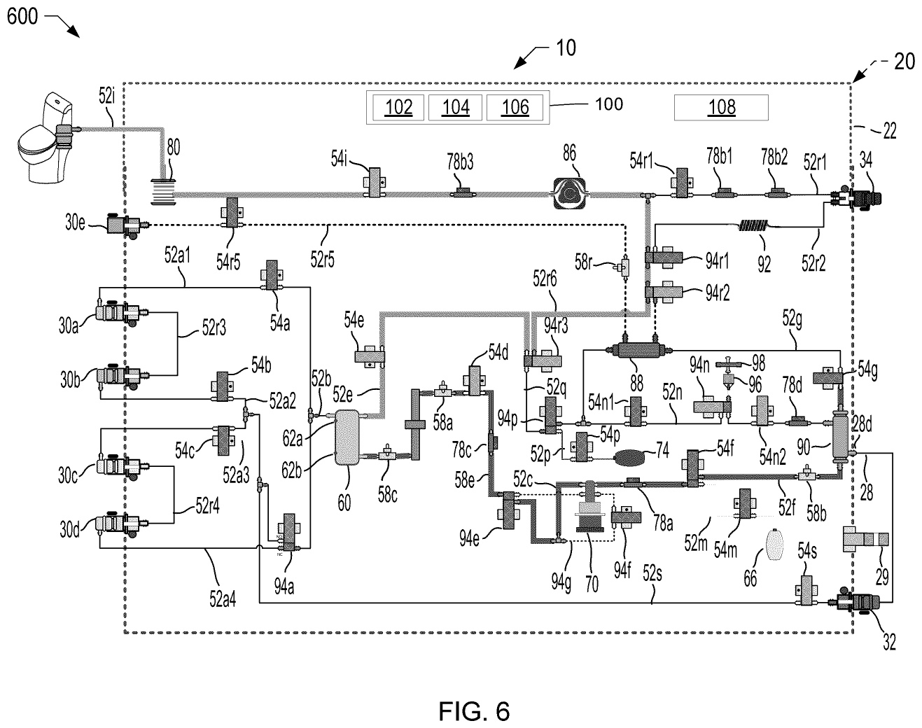

FIG. 6 illustrates a fourth fluid flow schematic 600 of one embodiment of the PD system 10. The fourth fluid flow schematic 600 illustrates fluid flow for an integrity test. For example, solution is introduced into the system 10 in reusable line 58e, reusable line 52c, reusable patient tubing 52f, and reusable patient tubing 56g while air occupies vent line 52e, reusable tubing 52r6, and drain tubing 52i. In the configuration represented by fluid flow schematic 600, an integrity of the PD system 10 and/or the ultrafilter 90 can be tested.

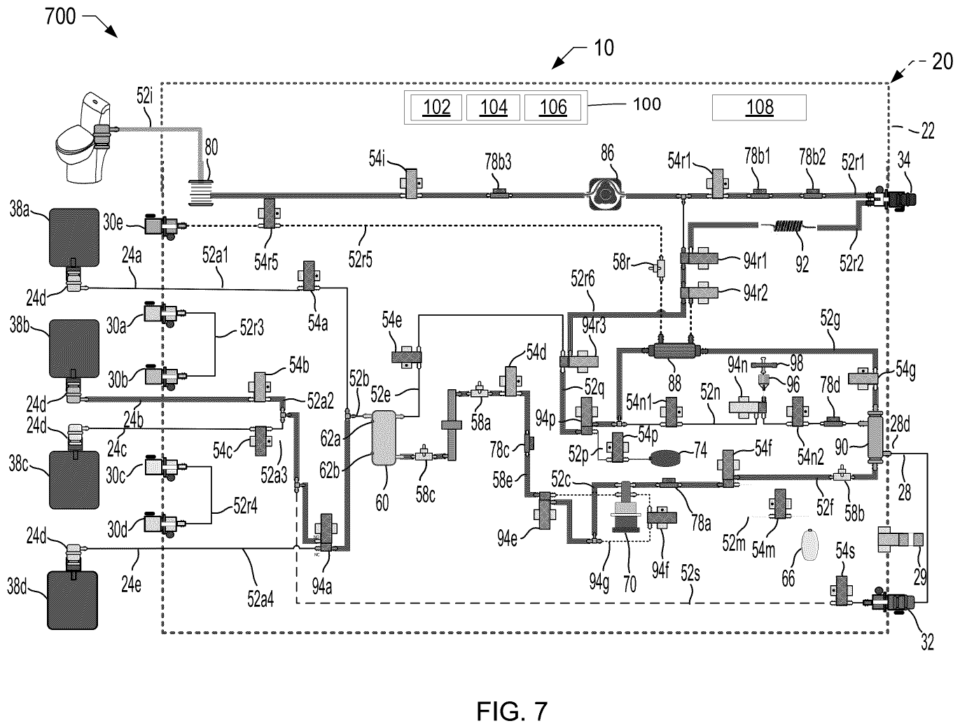

FIG. 7 illustrates a fifth fluid flow schematic 700 of one embodiment of the PD system 10. The fifth fluid flow schematic 700 illustrates fluid flow for flushing the solution lines. For example, in the first fluid flush of FIG. 7, solution is introduced from bag 38b and travels through reusable PD fluid line 24b, reusable supply tubing 52a2, reusable supply tubing 52b, reusable line 58e, reusable line 52c, reusable patient line 52f, reusable patent line 52g, reusable line 52r6, reusable line 52r2, reusable line 52r1, and drain line 52i in order to flush the aforementioned tubing.

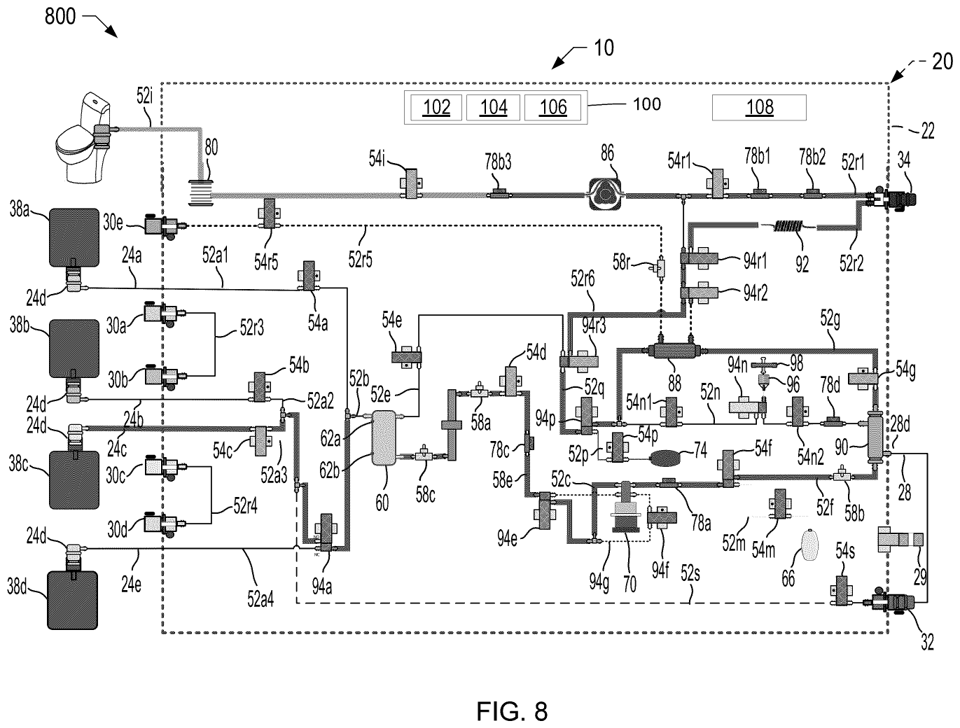

FIG. 8 illustrates a sixth fluid flow schematic 800 of one embodiment of the PD system 10. The sixth fluid flow schematic 800 illustrates a second example of fluid flow for flushing the solution lines. For example, in the second fluid flush of FIG. 8, solution is introduced from bag 38c and travels through reusable PD fluid line 24c, reusable supply tubing 52a3, reusable supply tubing 52b, reusable line 58e, reusable line 52c, reusable patient line 52f, reusable patent line 52g, reusable line 52r6, reusable line 52r2, reusable line 52r1, and drain line 52i in order to flush the aforementioned tubing.

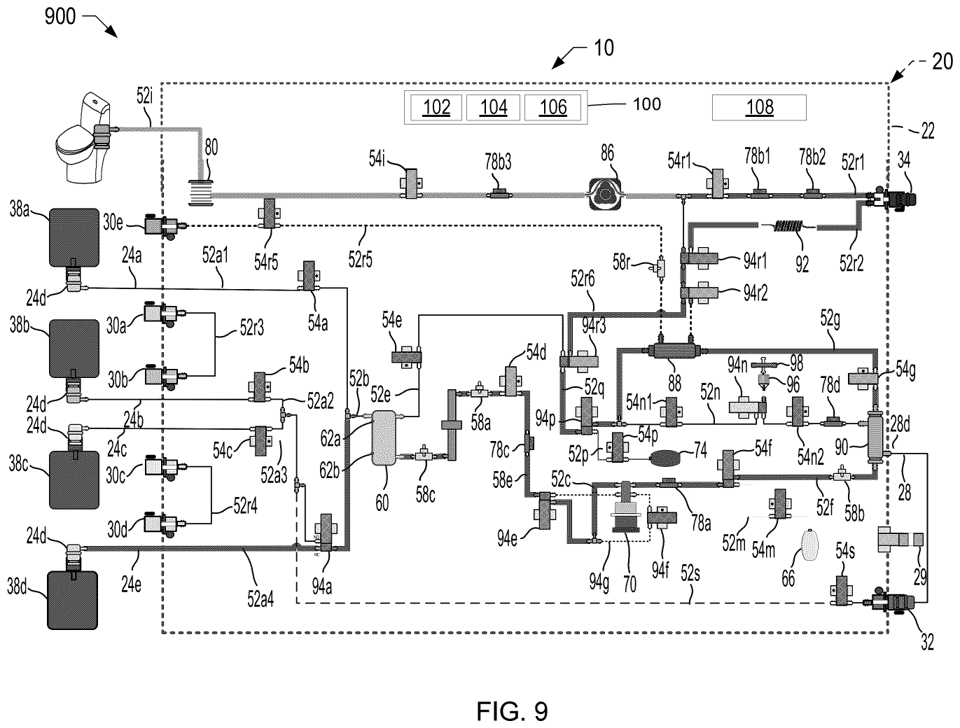

FIG. 9 illustrates a seventh fluid flow schematic 900 of one embodiment of the PD system 10. The seventh fluid flow schematic 900 illustrates a third example of fluid flow for flushing the solution lines. For example, in the third fluid flush of FIG. 9, solution is introduced from bag 38d and travels through reusable PD fluid line 24e, reusable supply tubing 52a4, reusable supply tubing 52b, reusable line 58e, reusable line 52c, reusable patient line 52f, reusable patent line 52g, reusable line 52r6, reusable line 52r2, and reusable line 52r1 in order to flush the aforementioned tubing.

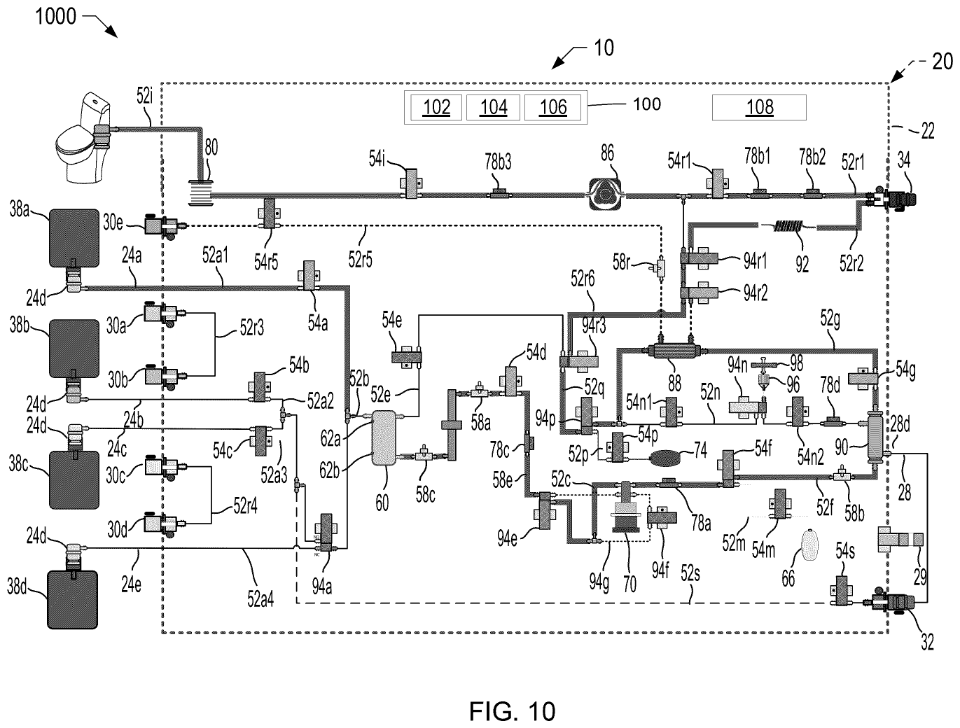

FIG. 10 illustrates an eighth fluid flow schematic 1000 of one embodiment of the PD system 10. The eighth fluid flow schematic 1000 illustrates a fourth example of fluid flow for flushing the solution lines. For example, in the fourth fluid flush of FIG. 10, solution is introduced from bag 38a and travels through reusable PD fluid line 24a, reusable supply tubing 52a1, reusable supply tubing 52b, reusable line 58e, reusable line 52c, reusable patient line 52f, reusable patent line 52g, reusable line 52r6, reusable line 52r2, reusable line 52r1, and drain line 52i in order to flush the aforementioned tubing.

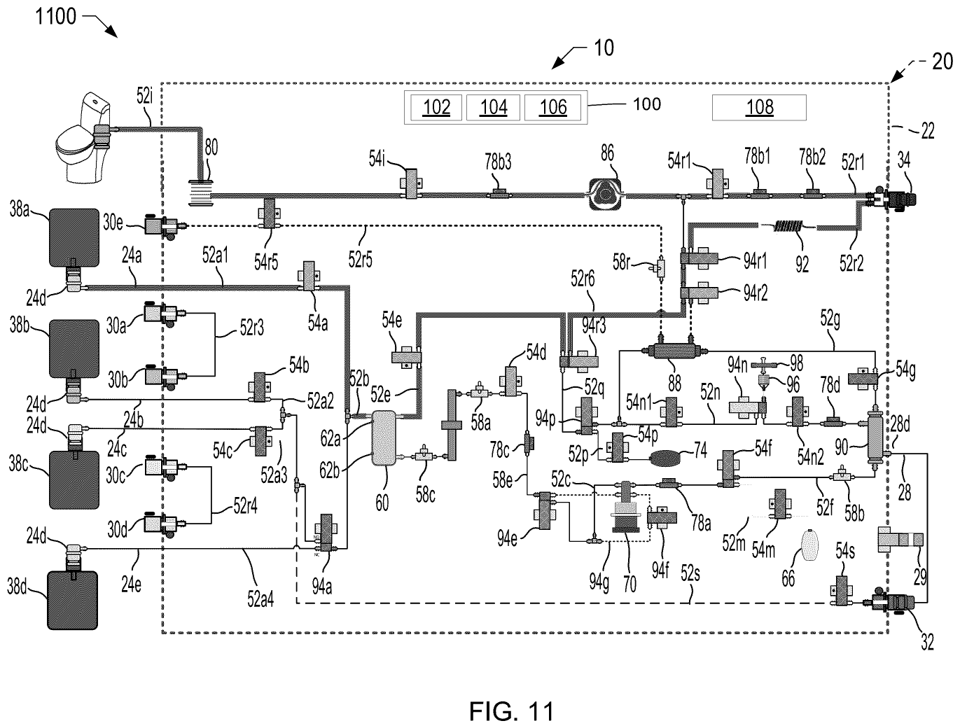

FIG. 11 illustrates a ninth fluid flow schematic 1100 of one embodiment of the PD system 10. The ninth fluid flow schematic 1100 illustrates an example of fluid flow for priming the air trap 60. For example, after the fluid flush of FIG. 10, the valve 58e may be opened to allow the fluid from bag 38a to flow through reusable line 52e and into reusable line 52r6. In this manner, the air trap 60 may be primed for use.

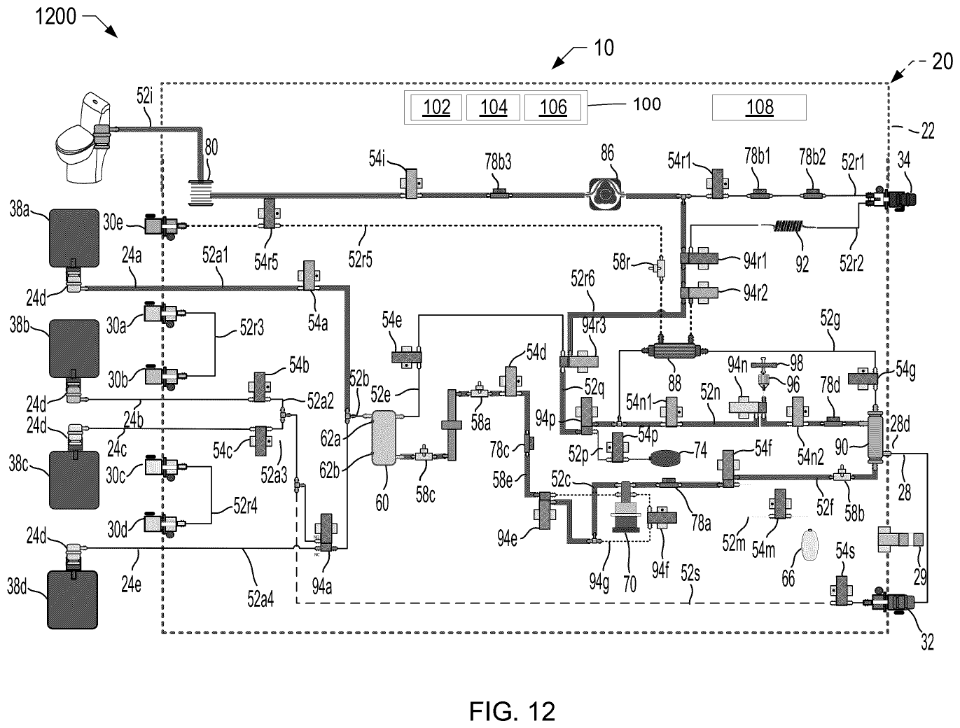

FIG. 12 illustrates a tenth fluid flow schematic 1200 of one embodiment of the PD system 10. The tenth fluid flow schematic 1200 illustrates an example of fluid flow for priming the ultrafilter 90. For example, after the priming of the air trap 60 of FIG. 11, the valves 54e and 94r1 may be closed so as to direct fluid flow through reusable patient line 52f and reusable patient line 52g in order to prime the ultrafilter 90.

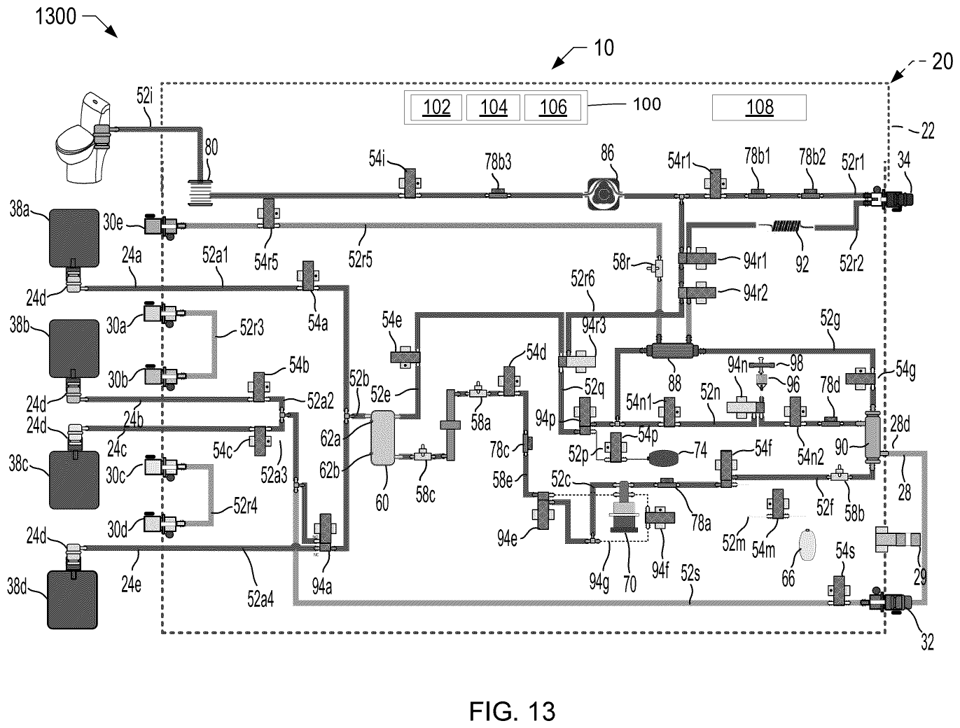

FIG. 13 illustrates an eleventh fluid flow schematic 1300 of one embodiment of the PD system 10. The eleventh fluid flow schematic 1300 illustrates an example of fluid flow after a completed flush. For example, after completing the flush procedure of FIGS. 3-12, reusable line 52s, recirculation line 52r3, and recirculation line 52r4 are filled with a low volume of air. Additionally, recirculation line 52r5 is filled with a low volume of heat disinfected solution. The remainder of the tubing or lines of the PD system 10 are filled with fluid from the bags 38a to 38d.

### Fill

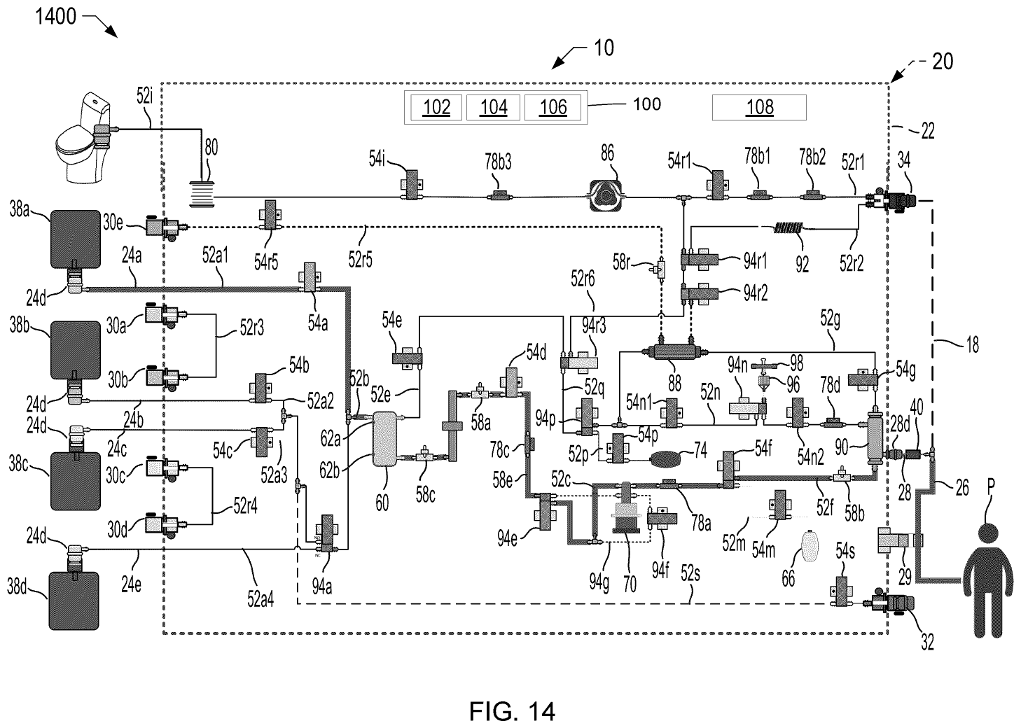

FIG. 14 illustrates a twelfth fluid flow schematic 1400 of one embodiment of the PD system 10. The twelfth fluid flow schematic 1400 illustrates an example of fluid flow during fill. For example, during a fill portion of a dialysis treatment, solution from the bag 38a is pumped from the bag 38a through reusable supply line 52a1, reusable supply line 52b, reusable line 58e, reusable line 52c, reusable patient line 52f, through the ultrafilter 90, patient line 28, disposable filter set 40, and patient's transfer set 26 into the patient P. In other examples of fill portions of dialysis treatment using the PD system 10, solution may be pumped instead or in addition to fluid from bags 38b, 38c, and/or 38d.

### Drain

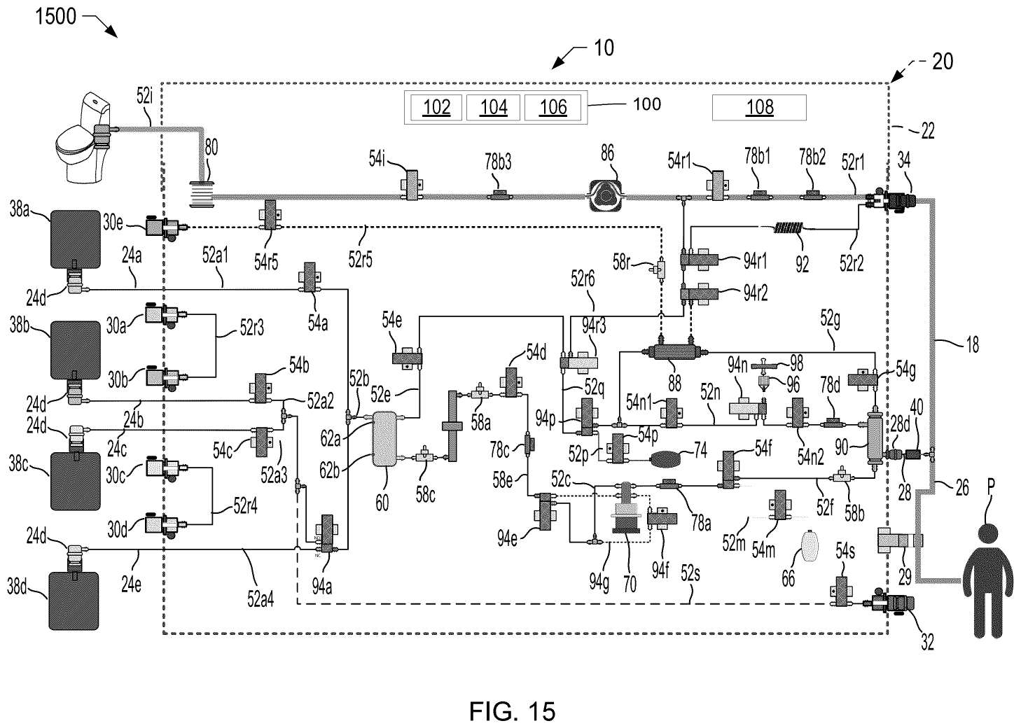

FIG. 15 illustrates a thirteenth fluid flow schematic 1500 of one embodiment of the PD system 10. The thirteenth fluid flow schematic 1500 illustrates an example of fluid flow during drain. For example, during a drain portion of a dialysis treatment, spent dialysate is removed from the patient P via the patient transfer set 26, reusable line 52r1, and drain line 52i into a drain. During a dialysis treatment, the fill portion of FIG. 14 and the drain portion of FIG. 15 may be repeated a number of times.

### Air Trap Fill

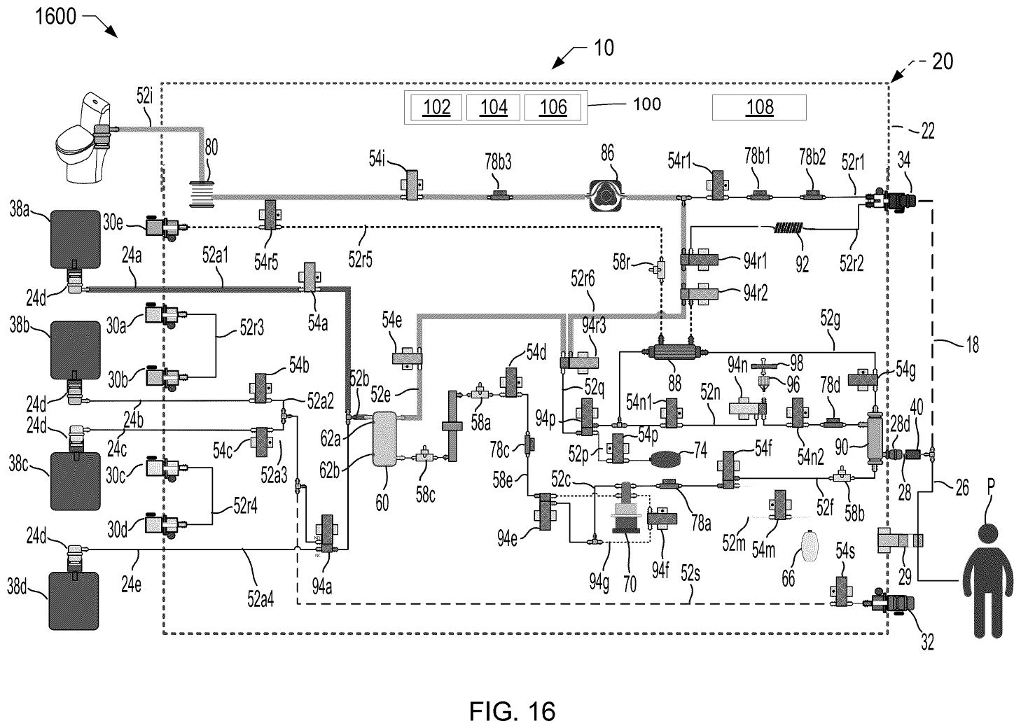

FIG. 16 illustrates a fourteenth fluid flow schematic 1600 of one embodiment of the PD system 10. The fourteenth fluid flow schematic 1600 illustrates an example of fluid flow during filling of the air trap. For example, after a drain portion of a dialysis treatment as described in conjunction with FIG. 15, it may be desirable to ensure the air trap 60 is filled with solution to maintain proper function. As shown in FIG. 16, the valve 54e may be opened and the valve 54d may be closed and fluid introduced from a supply source (e.g., the bag 38a) in order to fill the air trap 60.

### Tear-Down

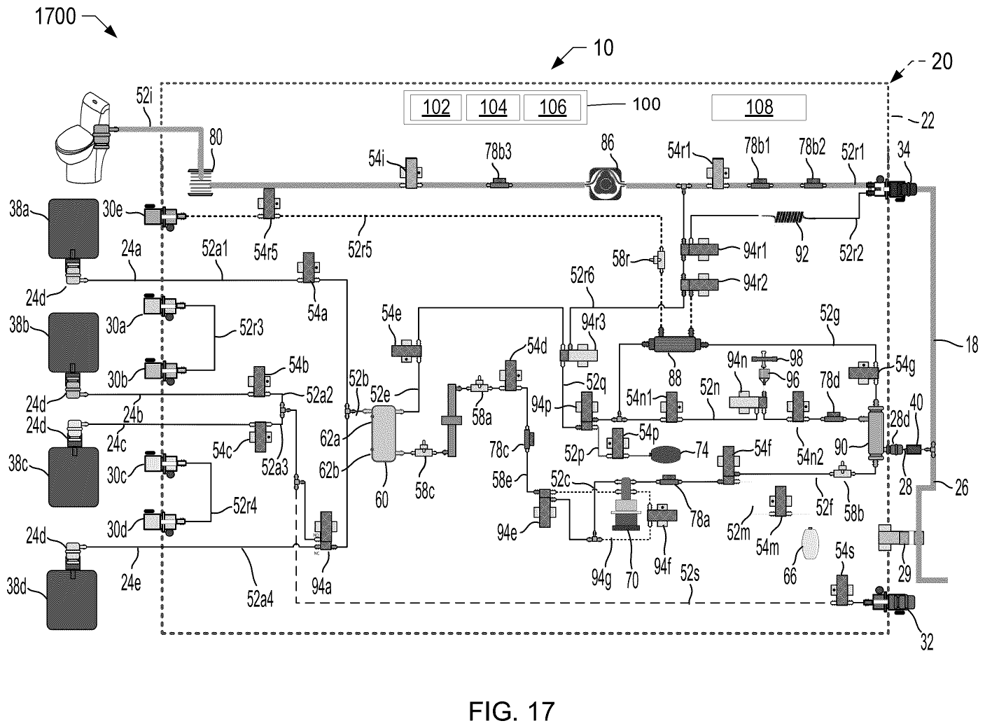

FIG. 17 illustrates a fifteenth fluid flow schematic 1700 of one embodiment of the PD system 10. The fifteenth fluid flow schematic 1700 illustrates an example of fluid flow during tear-down. For example, after a completed dialysis treatment, the patient P disconnects from the PD system 10. After disconnect, air remains in the reusable drain line 18, reusable line 52r1, and drain line 52i.

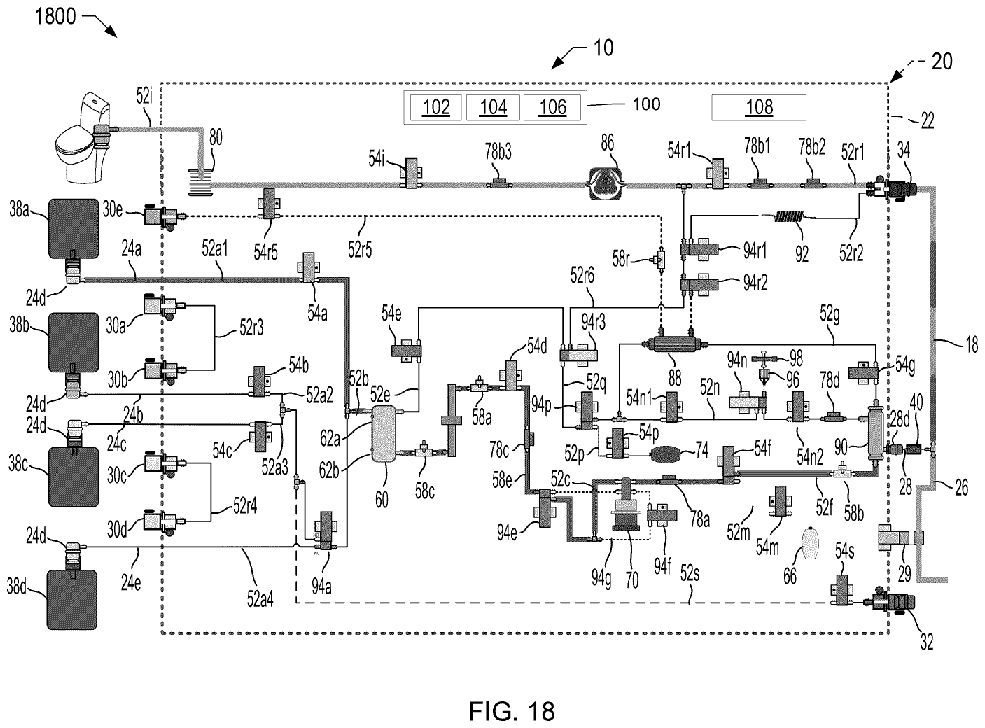

FIG. 18 illustrates a sixteenth fluid flow schematic 1800 of one embodiment of the PD system 10. The sixteenth fluid flow schematic 1800 illustrates a second example of fluid flow during tear-down. For example, following patient disconnect from the PD machine 10, any remaining fluid in the bags 38a to 38d may be pumped through the PD machine 10 to the drain.

### Disinfection

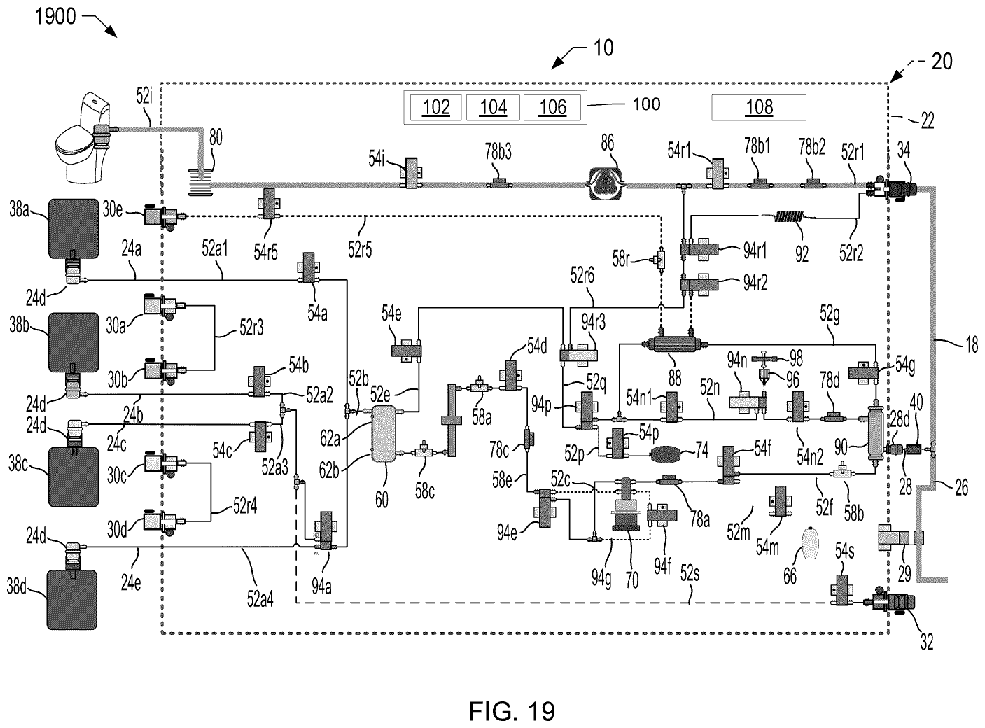

FIG. 19 illustrates a seventeenth fluid flow schematic 1900 of one embodiment of the PD system 10. The seventeenth fluid flow schematic 1900 illustrates an example of fluid flow for pre-disinfection tear-down. For example, after a completed dialysis treatment, the patient P disconnects from the PD system 10. After disconnect, air remains in the reusable drain line 18, reusable line 52r1, and drain line 52i.

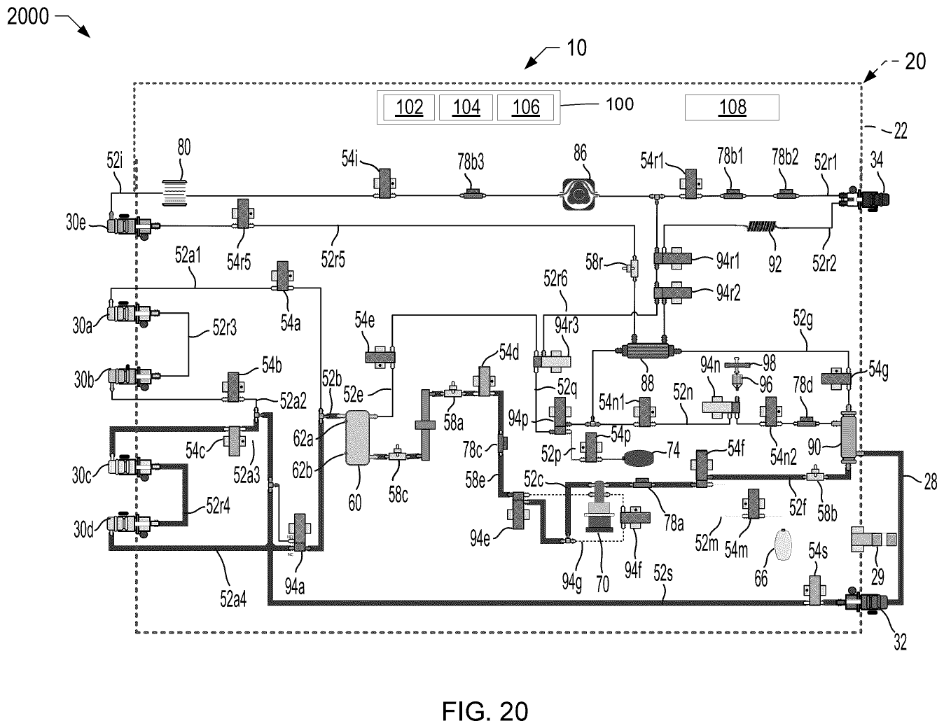

FIG. 20 illustrates an eighteenth fluid flow schematic 2000 of one embodiment of the PD system 10. The eighteenth fluid flow schematic 2000 illustrates an example of fluid flow during disinfection. During disinfection, the bags 38a to 38d are removed from the PD system 10. The example patient line 28 is attached to the connector 32, and the reusable supply lines 52a1, 52a2, 52a3, and 52a4 are connected to disinfection connectors 30a, 30b, 30c, and 30d respectively. In the example of FIG. 20, valves 54c and 94a are open to form a first disinfection circuit. Tubing and components in the first disinfection circuit may be disinfected using heat or other disinfection methods.

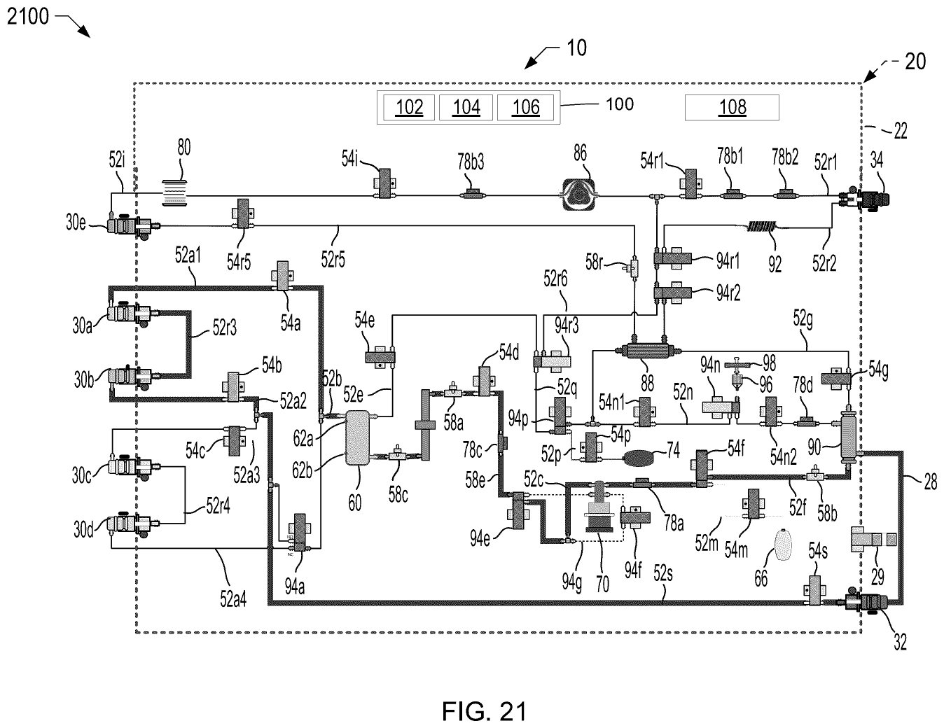

FIG. 21 illustrates a nineteenth fluid flow schematic 2100 of one embodiment of the PD system 10. The nineteenth fluid flow schematic 2100 illustrates a second example of fluid flow during disinfection. In the second disinfection example of FIG. 21, the example patient line 28 is attached to the connector 32, the reusable supply lines 52a1, 52a2, 52a3, and 52a4 are connected to disinfection connectors 30a, 30b, 30c, and 30d respectively, and valves 54b and 54a are open to form a second disinfection circuit. Tubing and components in the second disinfection circuit may be disinfected using heat or other disinfection methods.

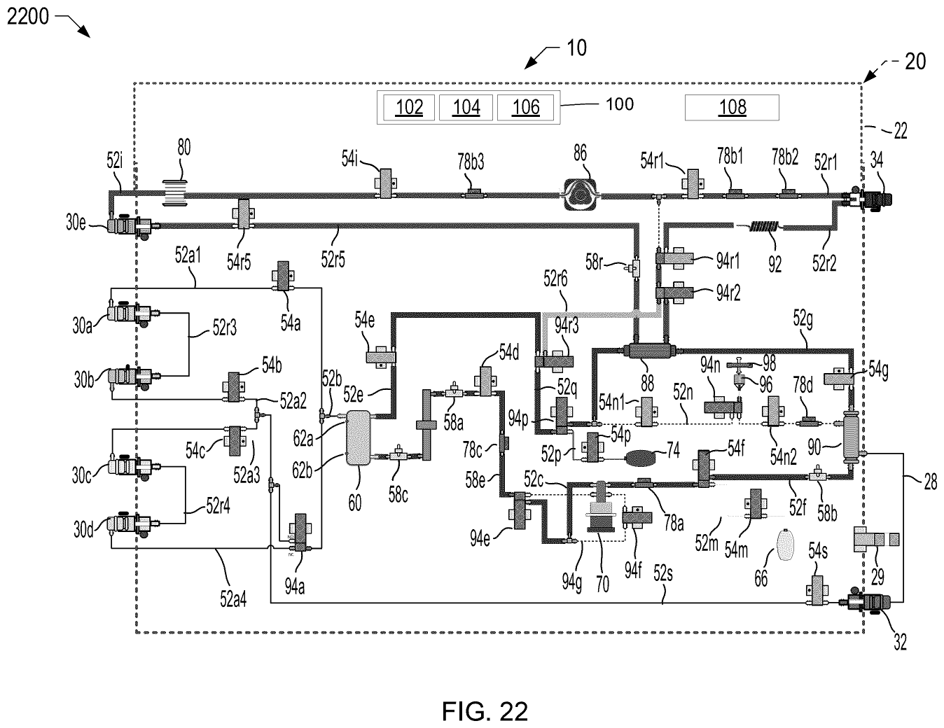

FIG. 22 illustrates a twentieth fluid flow schematic 2200 of one embodiment of the PD system 10. The twentieth fluid flow schematic 2200 illustrates a third example of fluid flow during disinfection. In the third disinfection example of FIG. 22, the example valves 54e, 54d, 54g, 54i, 54r1, 94r1, and 54r5 are open to form a third disinfection circuit. Tubing and components in the third disinfection circuit may be disinfected using heat or other disinfection methods.

### Filter Integrity Test Embodiments

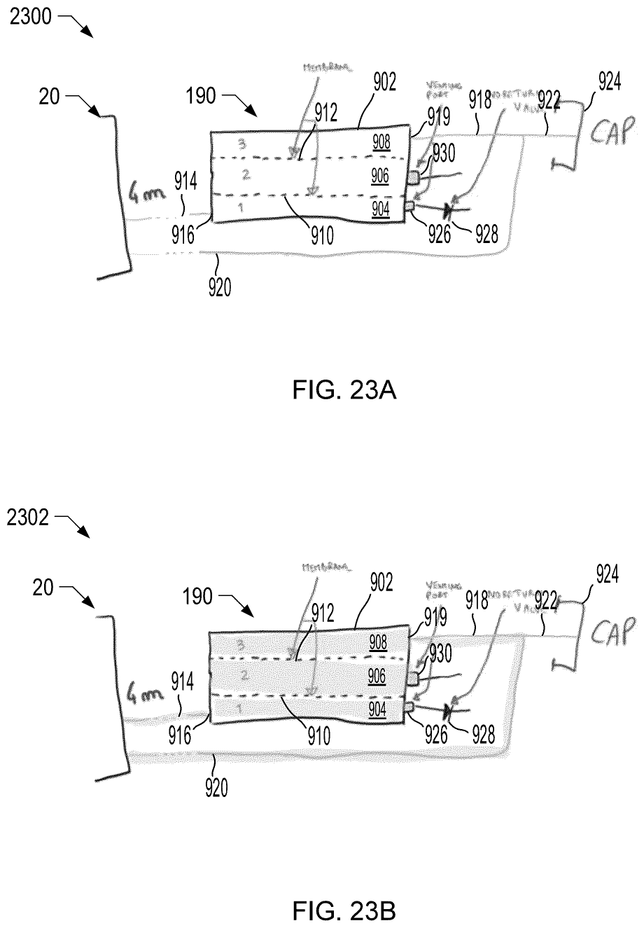

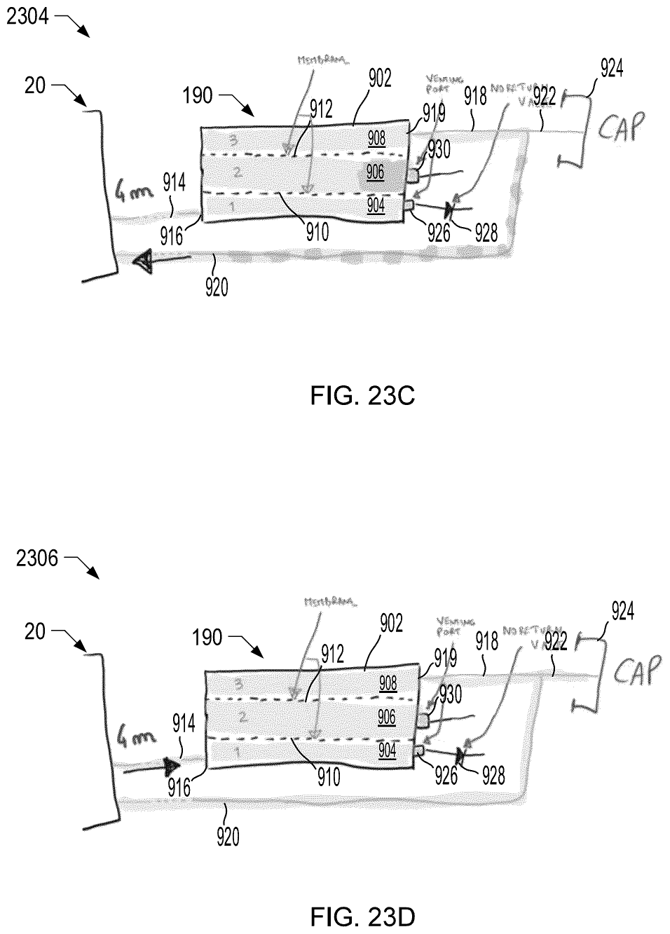

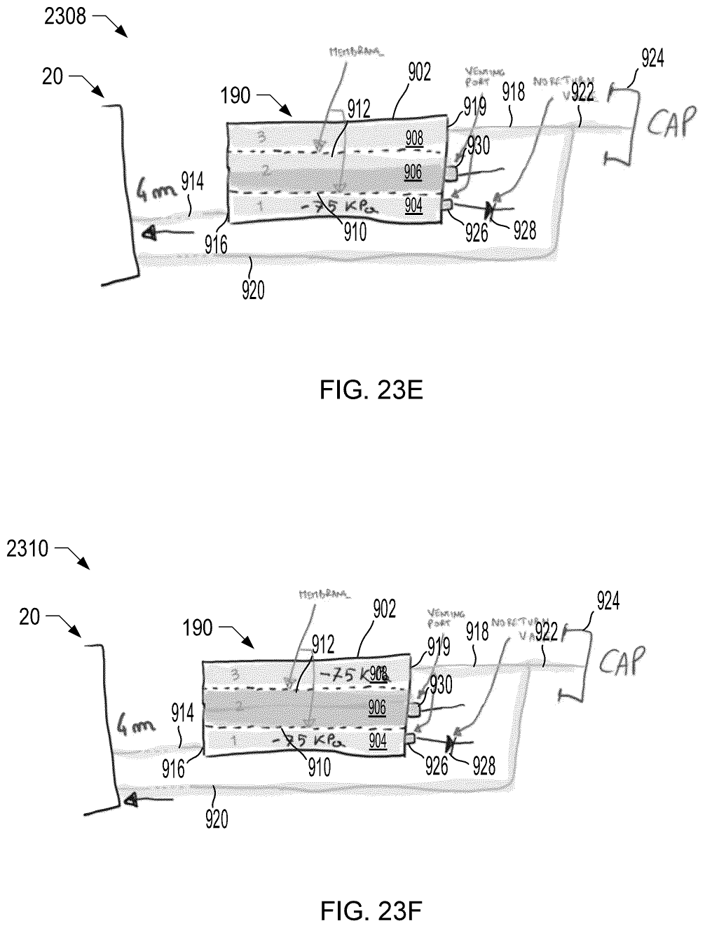

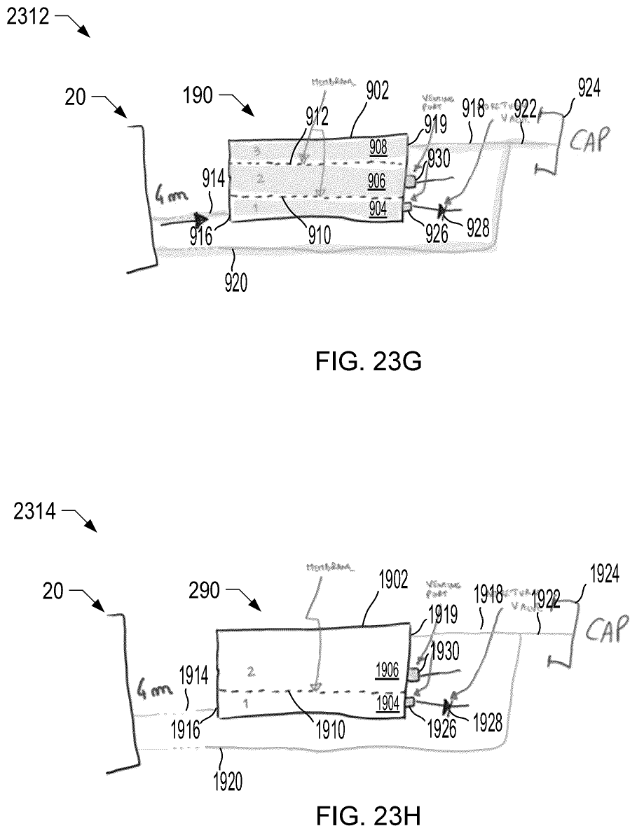

FIG. 23A is an illustration 2300 showing detail of an example filter 190. The example filter 190 may be an ultrafilter, a membrane filter, or a sterile membrane filter which may be used with the PD system 10. For example, the filter 190 may be a sterile membrane filter which comprises a portion of the disposable filter set 40 of the PD system 10. In another example, the filter 190 may be an ultrafilter such as the ultrafilter 90 of the PD system 10. The filter 190 may be used in the example PD system 10 in order to remove contaminants from the dialysis fluid. The example filter 190 removes contaminants from the dialysis fluid by passing the dialysis fluid through a series of one or more membranes capable of filtering out undesired contaminants having at least a certain size. As such, in order to effectively remove contaminants, the filter 190 must be free from broken fibers, holes, openings or other deformities in the membranes. Therefore, it is desirable to evaluate the integrity of the one or more membranes of the filter 190. In the example of FIG. 23A, the filter 190 is configured for conducting a filter integrity test.

The example filter 190 of FIG. 23A includes a filter housing 902. The example filter housing 902 contains a plurality of chambers separated by membranes. For example, the filter housing 902 includes a first outer chamber 904, a central chamber 906, and a second outer chamber 908. The example first outer chamber 904 and the example central chamber 906 are separated by a first membrane 910. The example central chamber 906 and the example second outer chamber 908 are separated by a second membrane 912. The example first membrane 910 and second membrane 912 are capable of allowing water and other particles smaller than a pore size of the membrane to pass through the membrane. The example first membrane 910 and second membrane 912 therefore exclude any particles larger than a pore size of the membrane from passing through. In some examples, the first membrane 910 and the second membrane 912 have the same pore size. In other examples, a pore size of the first membrane 910 is different than a pore size of the second membrane 912.

In some examples, the filter 190 may further include one or more supporting structures (not illustrated) separating the first outer chamber 904 and the central chamber 906 and/or one or more supporting structures separating the central chamber 906 and the second outer chamber 908. For example, a supporting structure may be placed on one or both sides of the first membrane 910 to provide mechanical stability to the first membrane. A supporting structure may also be placed on one or both sides of the second membrane 912 to provide mechanical stability to the second membrane. In some examples, the supporting structure(s) may provide mechanical stability during filter integrity testing.

The example filter 190 of FIG. 23A is connected to the PD machine 20. For example, a supply line 914 provides fluid connection between the PD machine 20 and an inlet 916 of the example filter 190. Additionally, an example drain line 918 provides fluid connection between an outlet 919 of the example filter 190 and the PD machine 20. The example outlet 919 is connected to the second outer chamber 908 of the filter 190. In the example of FIG. 23A, the drain line 918 is split downstream of the filter 190 into a return portion 920 and a cap portion 922. The example return portion 920 of the drain line 918 provides the fluid connection to the PD machine 20. The example cap portion 922 terminates at a cap 924.

The example first outer chamber 904 includes the inlet 916 at a first end and a first venting port 926 at a second end. The example first venting port 926 allows air to flow from the first outer chamber 904 to outside of the housing 902 of the filter 190. The example first venting port 926 is coupled with an example no return valve 928. The example no return valve (e.g., check valve, non-return valve, NRV) 928 prevents air from flowing from outside of the housing 902 to inside of the first outer chamber 904. The example central chamber 906 includes a second venting port 930. The second venting port 930 is not coupled with a no return valve. Thus, the second venting port 930 allows air to flow from inside the central chamber 906 to outside the housing 902 and from outside the housing 902 to inside the central chamber 906. The example PD machine 20 includes a control unit (e.g., the control unit 100) to control one or more pumps of the PD machine 20 and read one or more sensors of the PD machine 20 during filter integrity testing.

FIG. 23B illustrates a first phase 2302 of a filter integrity test of the filter 190 of the PD system 10. In the example first phase 2302 of the filter integrity test, the filter 190 is fully primed with fresh fluid. For example, a pump of the PD machine 20 causes fluid to flow through the supply line 914 and into the filter 190 such that the first outer chamber 904, the central chamber 906, and the second outer chamber 908 are filled with the fluid. As the fluid is added to the filter 190, the first membrane 910 and the second membrane 912 are wetted. Further, the fluid flows out the outlet 919 of the filter 190 into the drain line 918 and the return portion 920 of the drain line such that the return portion 920 of the drain line is also filled with fluid. During the first phase 2302 of the filter integrity test, the cap portion 922 of the drain line 918 remains empty (e.g., not filled with fluid).

FIG. 23C illustrates a first portion 2304 of a second phase of the filter integrity test of the filter 190. The first portion 2304 of the second phase of the filter integrity test commences upon completion of the first phase 2302 of the filter integrity test. In the second phase of the filter integrity test, air is removed from the cap portion 922 of the drain line. In the example first portion 2304 of the second phase of the filter integrity test, a pump of the PD machine 20 removes a portion of fluid the filter 190 such that a negative pressure is formed in the cap portion 922 of the drain line 918. For example, as the pump of the PD machine 20 applies a negative pressure to the fluid in the drain line 918, the second venting port 930 allows air to flow into the central chamber 906 of the filter 190. As a result, a portion of the fluid from the filter 190 is removed and the drain line 918 including the return portion 920 and the cap portion 922 experience a negative pressure. In some examples, the negative pressure in the cap portion 922 is approximately −75 kPa. In other examples, the negative pressure in the cap portion 922 may be more or less than −75 kPa while still being a negative pressure.

FIG. 23D illustrates a second portion 2306 of the second phase of the filter integrity test of the filter 190. The example second portion 2306 of the second phase of the filter integrity test commences upon completion of the first portion 2304 of the second phase of the filter integrity test. In the example second portion 2306 of the second phase of the filter integrity test, the fluid in the filter 190 is re-equalized, minimizing the air in the cap portion 922 of the drain line 918. For example, a pump of the PD machine 20 causes fluid to flow through the supply line 914 and in to the filter 190 such that the pressure inside the filter 190 is approximately the same as the pressure outside of the filter 190. As a result of the re-equalizing, the cap portion 922 of the drain line 918 is filled with fluid.

FIG. 23E illustrates a third phase 2308 of the filter integrity test of the filter 190. In the example third phase 2308 of the filter integrity test, an integrity of the first membrane 910 is tested. The example third phase 2308 of the filter integrity test commences upon completion of the second phase of the filter integrity test. In some examples, the integrity of the first membrane 910 is tested using a negative pressure test. In other examples, the integrity of the first membrane 910 is tested using a positive pressure test.

To test the integrity of the first membrane 910 using a negative pressure test, the control unit 100 causes a pump of the PD machine 20 to remove fluid from the filter 190 through the supply line 914. As the fluid is removed from the filter 190 through the supply line 914, air enters the central chamber 906 of the filter 190 through the second venting port 930. In examples disclosed herein, air cannot pass through the first membrane 910 after the first membrane 910 is wetted (e.g., during the priming of the first phase 2302). Thus, as a result, a pressure inside the first outer chamber 904 of the filter 190 is reduced. For example, the pump may remove fluid until the pressure inside the first outer chamber 904 reaches a threshold. In some examples, the pressure threshold is −75 kPa. In other examples, the pressure threshold may be more or less than −75 kPa while still being a negative pressure.

To test the integrity of the first membrane 910 using a positive pressure test, a pump of the PD machine 20 pumps air into the filter 190 via the supply line 914. In examples disclosed herein, air cannot pass through the first membrane 910 after the first membrane 910 is wetted (e.g., during the priming of the first phase 2302). Thus, as the air is added to the filter 190, a pressure inside the first outer chamber 904 is increased. For example, the pump may add air until the pressure inside the first outer chamber 904 reaches a threshold. In some examples, the pressure threshold is 75 kPa. In other examples, the pressure threshold may be more or less than 75 kPa while still being a positive pressure.

The example PD machine 20 includes one or more pressure sensors (e.g., pressure sensors 78a to 78d) for monitoring the pressure of the filter 190. After an output from the pressure sensor is indicative that the pressure threshold in the filter 190 has been reached, the pump stops removing fluid or air from the filter 190 or adding fluid or air to the filter 190. At this time, the pressure sensor in conjunction with the control unit 100 monitors the pressure in the filter 190 for a period of time in order to determine when the pressure in the filter 190 is approximately stable (e.g., not changing, not changing more than 1%, not changing more than 0.1%, etc.). In some examples, the period of time the pressure is monitored is approximately 90 seconds. In other examples, the period of time the pressure is monitored is any other time sufficient to determine if the pressure in the filter 190 is approximately stable.

When the control unit 100 determines that the pressure in the filter 190 is not approximately stable during this period of time, it is determined that the first membrane 910 fails the integrity test. For example, when the first membrane 910 has one or more openings greater than 4 microns in diameter, the first membrane 910 will fail the integrity test. If the first membrane 910 fails the integrity test, the test is completed at the third phase 2308 of the integrity test. When the control unit 100 determines that the pressure in the filter 190 is approximately stable during this period of time, it is determined that the first membrane 910 passes the integrity test. For example, it is determined that the first membrane 910 does not have any opening greater than 4 microns in diameter. If the first membrane 910 passes the integrity test, the integrity test moves on to the fourth phase of the test as described below.

FIG. 23F illustrates a fourth phase 2310 of the filter integrity test of the filter 190. In the example fourth phase 2310 of the filter integrity test, an integrity of the second membrane 912 is tested. The example fourth phase 2310 of the filter integrity test commences upon completion of the third phase 2308 of the filter integrity test. In some examples, the integrity of the second membrane 912 is testing using a negative pressure test. In other examples, the integrity of the second membrane 912 is tested using a positive pressure test. In some examples, the type of test (e.g., negative pressure or positive pressure) for the second membrane 912 is the same as the type of integrity test used for the first membrane 910 at the third phase 2308.

To test the integrity of the second membrane 912 using a negative pressure test, a pump of the PD machine 20 removes fluid from the filter 190 through the drain line 918. As the fluid is removed from the filter 190 through the drain line 918, air enters the central chamber 906 of the filter 190 through the second venting port 930. In examples disclosed herein, air cannot pass through the second membrane 912 after the second membrane 912 is wetted (e.g., during the priming of the first phase 2302). Thus, as a result, a pressure inside the second outer chamber 908 of the filter 190 is reduced. For example, the pump may remove fluid until the pressure inside the second outer chamber 908 reaches a threshold. In some examples, the pressure threshold is −75 kPa. In other examples, the pressure threshold may be more or less than −75 kPa while still being a negative pressure.

To test the integrity of the second membrane 912 using a positive pressure test, a pump of the PD machine 20 pumps air into the filter 190 via the drain line 918. In examples disclosed herein, air cannot pass through the second membrane 912 after the second membrane 912 is wetted (e.g., during the priming of the first phase 2302). Thus, as the air is added to the filter 190, a pressure inside the second outer chamber 908 is increased. For example, the pump may add air until the pressure inside the second outer chamber 908 reaches a threshold. In some examples, the pressure threshold is 75 kPa. In other examples, the pressure threshold may be more or less than 75 kPa while still being a positive pressure.

During this time, a pressure sensor of the PD machine 20 operates with the control unit 100 to monitor the pressure in the filter 190. After the control unit 100 determines that the pressure threshold in the filter 190 is reached, the control unit 100 causes the pump to stop removing or adding fluid or air from the filter 190. At this time, the pressure sensor operating with the control unit 100 monitors the pressure in the filter 190 for a period of time in order to determine when the pressure in the filter 190 is approximately stable. In some examples, the period of time the pressure is monitored is approximately 90 seconds. In other examples, the period of time the pressure is monitored is any other time sufficient to determine if the pressure in the filter 190 is approximately stable.

The control unit 100 uses an output from the pressure sensor to determine that the pressure in the filter 190 is not approximately stable during this period of time. The control unit 100 accordingly is configured to determined that the second membrane 912 fails the integrity test. For example, if the second membrane 912 has one or more openings greater than 4 microns in diameter, the second membrane 912 will fail the integrity test. When the control unit 100 determines that the pressure in the filter 190 is approximately stable during this period of time, it is determined that the second membrane 912 passes the integrity test. For example, it is determined that the second membrane 912 does not have any opening greater than 4 microns in diameter. If both the first membrane 910 and the second membrane 912 pass the integrity test, it is found that the filter 190 passes the filter integrity test.

After the filter integrity test is performed, the control unit 100 of the PD machine 20 may communicate the determined integrity of the first membrane and/or the second membrane. For example, the control unit 100 may cause the integrity status(es) to be displayed on a user interface of the PD machine 20. In another example, the control unit 100 may send data (e.g., wirelessly or by a wired connection) corresponding to the results of the integrity test to another device (or a server) for access by the device and/or a user of the device.

FIG. 23G illustrates a fifth phase 2312 of the filter integrity test of the filter 190. In the example fifth phase 2312 of the filter integrity test, the filter 190 is re-equalized. The example fifth phase 2312 of the filter integrity test commences upon completion of the fourth phase 2310 of the filter integrity test. In the example where the integrity test on the first membrane 910 and the second membrane 912 is a negative pressure test, the filter 190 is re-filled with fluid during the fifth phase 2312 in order to re-equalize the filter 190. In the example where the integrity test on the first membrane 910 and the second membrane 912 is a positive pressure test, air is removed from the filter 190 during the fifth phase 2312 in order to re-equalize the filter 190.

To re-fill the filter 190 with fluid following a negative pressure test, a pump of the PD machine 20 causes fluid to enter the filter 190 through the supply line 914. This fluid displaces air that entered the central chamber 906 of the filter 190 during the third phase 2308 and/or the fourth phase 2310 of the filter integrity test. As a result, the central chamber 906 of the filter 190 is filled with fluid and pressure inside the filter 190 is equalized to approximately the same as the pressure outside the filter 190.

To remove air from the filter 190 following a positive pressure test, a pump of the PD machine 20 causes air to be pumped out of the filter 190 through the supply line 914. In some examples, a pump of the PD machine 20 additionally or alternatively causes air to be pumped out of the filter 190 through the drain line 918. As a result, the pressure inside the filter 190 (e.g., the pressure inside the first outer chamber 904 and the second outer chamber 908) is equalized to approximately the same as the pressure outside the filter 190.

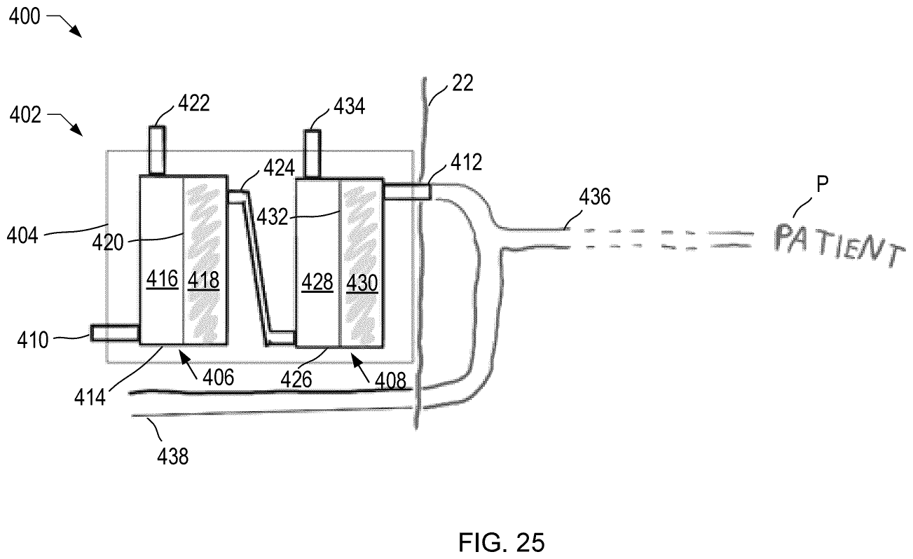

While the example filter 190 of FIGS. 23A-23G is illustrated with a single housing 902, three chambers (the first outer chamber 904, the central chamber 906, and the second outer chamber 908), and two membranes (the first membrane 910 and the second membrane 912), it should be understood that the filter integrity test described in FIGS. 23B-23G may be performed using alternative filter designs. For example, a filter having two housings, each housing having two chambers and one membrane as shown in FIG. 25 described below, may also be evaluated using the filter integrity test described herein.

FIG. 23H is an illustration 2314 showing further detail of a second example filter 290 which may be used with the PD system 10. The example second filter 290 may be an ultrafilter, a membrane filter, or a sterile membrane filter which may be used with the example PD system 10 in order to remove contaminants from the dialysis fluid. For example, the second example filter 290 may be a sterile membrane filter which comprises a portion of the disposable filter set 40 of the PD system 10. In another example, the second example filter 290 may be an ultrafilter such as the ultrafilter 90 of the PD system 10. The second example filter 290 removes contaminants from the dialysis fluid by passing the dialysis fluid through a membrane capable of filtering out undesired contaminants having at least a certain size. In contrast to example filter 190 of FIGS. 23A-23G, the second example filter 290 has a single membrane 1910. The example filter integrity test described in FIGS. 23B-23G may be used to evaluate the integrity of the single membrane 1910 of the second example filter 290.

The second example filter 290 of FIG. 23A includes a filter housing 1902. The example filter housing 1902 contains a first chamber 1904 and a second chamber 1906 separated by the single membrane 1910. The example single membrane 1910 is capable of allowing water and other particles smaller than a pore size of the membrane to pass through the membrane. The example single membrane 1910 therefore excludes any particles larger than a pore size of the membrane from passing through. In examples where the filter 190 is an ultrafilter, the single membrane 1910 may comprise a plurality of hollow fibers, the first chamber 1904 may be a concentrate side of the filter 290 and the second chamber 1906 may be a filtrate side of the filter 290.

In some examples, the second example filter 290 may further include one or more supporting structures (not illustrated) separating the first chamber 1904 and the second chamber 1906. For example, a supporting structure may be placed on one or both sides of the single membrane 1910 to provide mechanical stability to the single membrane 1910. In some examples, the supporting structure(s) may provide mechanical stability during filter integrity testing.

The second example filter 290 of FIG. 23H is connected to the PD machine 20. For example, a supply line 1914 provides fluid connection between the PD machine 20 and an inlet 1916 of the second example filter 290. Additionally, an example drain line 1918 provides fluid connection between an outlet 1919 of the second example filter 290 and the PD machine 20. The example outlet 1919 is connected to the second chamber 1906 of the second example filter 290. In the example of FIG. 23H, the drain line 1918 is split downstream of the second example filter 290 into a return portion 1920 and a cap portion 1922. The example return portion 1920 of the drain line 1918 provides the fluid connection to the PD machine 20. The example cap portion 1922 terminates at a cap 1924.

The example first chamber 1904 includes the inlet 1916 at a first end and a first venting port 1926 at a second end. The example first venting port 1926 allows air to flow from the first chamber 1904 to outside of the housing 1902 of the second example filter 290. The example first venting port 1926 is coupled with an example no return valve 1928. The example no return valve (e.g., check valve, non-return valve, NRV) 1928 prevents air from flowing from outside of the housing 1902 to inside of the first chamber 1904. The example second chamber 1906 includes a second venting port 1930. The second venting port 1930 is not coupled with a no return valve. Thus, the second venting port 1930 allows air to flow from inside the second chamber 1906 to outside the housing 1902 and from outside the housing 1902 to inside the second chamber 1906. The example PD machine 20 includes a control unit (e.g., the control unit 100) to control one or more pumps of the PD machine 20 and read one or more sensors of the PD machine 20 during filter integrity testing.

To test the integrity of the single membrane 1910, at least a portion of the filter integrity test described in connection with FIGS. 23B-23G may be performed. For example, the second example filter 290 may be fully primed with fresh fluid, as illustrated in FIG. 23B. Subsequently, a pump of the PD machine 20 may remove a portion of fluid from the second example filter 290 such that a negative pressure is formed in the cap portion 1922 of the drain line 1918, as illustrated in FIG. 23C. Then, the fluid in the second example filter 190 may be re-equalized, minimizing the air in the cap portion 1922 of the drain line 1918, as illustrated in FIG. 23D.

In a subsequent phase, an integrity of the single membrane 1910 is tested. The integrity of the single membrane 1910 may be tested using a negative pressure test or a positive pressure test. To test the integrity of the single membrane 1910 using a negative pressure test, a pump of the PD machine 20 may remove fluid from the second example filter 290 through the supply line 1914. As the fluid is removed from the second example filter 290 through the supply line 1914, air enters the second chamber 1906 through the second venting port 1930. As a result, a pressure inside the first chamber 1904 of the second example filter 290 may be reduced. For example, the pump may remove fluid until the pressure inside the first chamber 1904 reaches a threshold. In some examples, the pressure threshold is −75 kPa. In other examples, the pressure threshold may be more or less than −75 kPa while still being a negative pressure.

To test the integrity of the single membrane 1910 using a positive pressure test, a pump of the PD machine 20 may pump fluid into the second example filter 290 via the supply line 1914. As a result, a pressure inside the first chamber 1904 of the second example filter 290 may be increased. For example, the pump may add fluid until the pressure inside the first chamber 1904 reaches a threshold. In some examples, the pressure threshold is 75 kPa. In other examples, the pressure threshold may be more or less than 75 kPa while still being a positive pressure.

After the control unit 100 uses an output from the pressure sensor of the PD machine 20 to determine that the pressure threshold in the second example filter 290 has been reached, the control unit 100 causes the pump to stop removing fluid from the second example filter 290. At this time, the pressure sensor operates with the control unit 100 to monitor the pressure in the second example filter 290 for a period of time in order to determine if the pressure in the second example filter 290 is approximately stable (e.g., not changing, not changing more than 1%, not changing more than 0.1%, etc.). In some examples, the period of time the pressure is monitored is approximately 90 seconds. In other examples, the period of time the pressure is monitored is any other time sufficient to determine if the pressure in the ultrafilter is approximately stable.

When the control unit 100 determines that the pressure in the second example filter 290 is not approximately stable during this period of time, it is determined that the single membrane 1910 fails the integrity test. For example, if the single membrane 1910 has one or more openings greater than 4 microns in diameter, the single membrane 1910 will fail the integrity test. When the control unit 100 determines that the pressure in the second example filter 290 is approximately stable during this period of time, it is determined that the single membrane 1910 passes the integrity test. For example, it is determined that the single membrane 1910 does not have any opening greater than 4 microns in diameter. If the single membrane 1910 passes the integrity test, it is found that the second example filter 290 passes the filter integrity test.

After the filter integrity test is performed, the control unit of the PD machine 20 may communicate the determined integrity of the single membrane 1910. For example, the control unit 100 may cause the integrity status to be displayed on a user interface of the PD machine 20. In another example, the control unit 100 may transmit data (e.g., wirelessly or by a wired connection) corresponding to the results of the integrity test to another device or server for access by the device and/or a user of the device. After the filter integrity is determined, the second example filter 290 may be re-filled with fluid as illustrated in FIG. 23G.

### Internal Flush Recirculation Embodiment

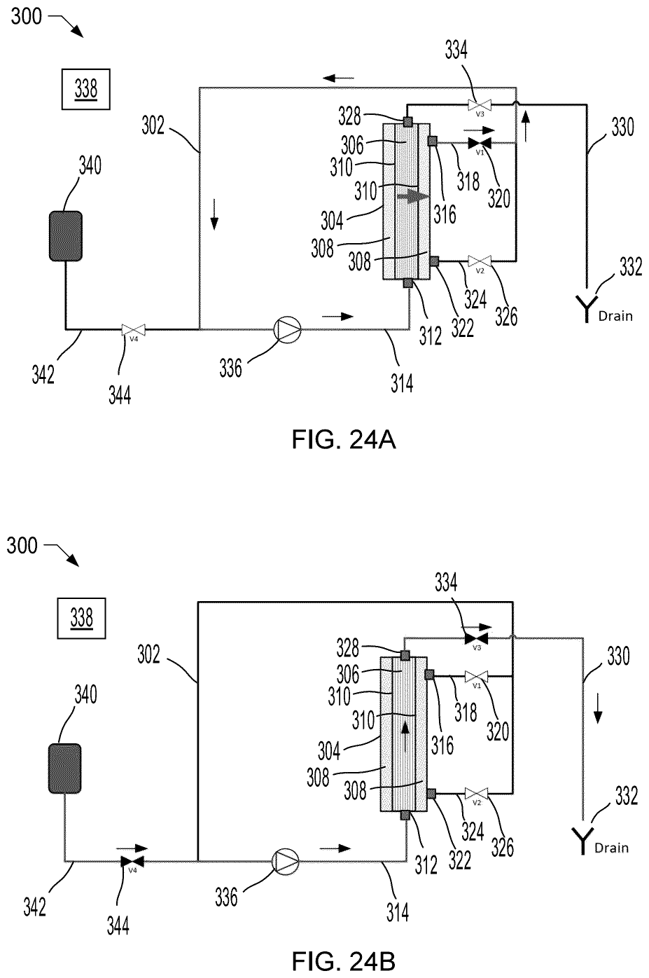

FIGS. 24A-24B illustrate an embodiment of a medical fluid system 300, which may be used to recirculate and flush fluid through the system 300. The example recirculation and flush process may be performed after a disinfection process (e.g., the disinfection process of FIGS. 20-22) is performed which may generate target molecules within the system 300.

FIG. 24A is a schematic of one embodiment of the medical fluid system 300 of the present illustrating a recirculation circuit 302. The example recirculation circuit 302 may be used to circulate a fixed volume of fluid through the system 300 through the filter 304 a number of times in order to increase the number of molecules that are size excluded by the membrane. In some examples, the system 300 may be substantially similar to the system 10 of FIG. 1. In some examples, the filter 304 may be the ultrafilter 90 of FIG. 1. In other examples, the filter 304 may be a different ultrafilter or a different type of filter (e.g., a high cut-off filter).

The example filter 304 of FIG. 24A includes a concentrate portion 306 and one or more filtrate portions 308. The example filter 304 may be substantially cylindrical such that the two filtrate portions 308 illustrated in the cross-sectional view of FIG. 24A represent a single, cylindrical filtrate portion 308. The example filter 304 of FIG. 24A further includes one or more membranes 310 which separate the concentrate portion 306 and the filtrate portion 308. The example filter may be substantially cylindrical such that the two membranes 310 illustrated in the cross-sectional view of FIG. 24A represent a single, cylindrical membrane 310. The example one or more membranes 310 are configured to allow a fluid (e.g., a solution) to pass from the concentrate portion 306 of the filter 304 to the filtrate portion 308 of the filter 304 while excluding molecules of a target size or greater from passing through the membrane 310. As such, a concentration of target molecules in the concentrate portion 306 is increased while a concentration of target molecules in the filtrate portion 308 is decreased. Example molecules that may be excluded by the membrane 310 include endotoxin units (EU) and colony-forming units (CFU).

The example filter 304 of FIG. 24A includes an inlet 312 connected to a first end of the concentrate portion 306 of the filter 304. The example inlet 312 provides fluid communication between the concentrate portion 306 of the filter 304 and a reusable filter supply line 314. The example reusable filter supply line 314 comprises a portion of the recirculation circuit 302 proximate to the inlet 312 of the filter 304. The example filter 304 further includes a first outlet 316 connected to the filtrate portion 308 of the filter. The example first outlet 316 provides fluid communication between the filtrate portion 308 of the filter 304 and a first filter outlet line 318. The example first filter outlet line 318 provides fluid communication between the first outlet 316 and the recirculation circuit 302. The example first filter outlet line 318 includes a first valve 320 which controls flow of fluid from the first outlet 316 into the recirculation circuit 302. The example filter 304 further includes a second outlet 322 connected to the filtrate portion 308 of the filter. The example second outlet 322 provides fluid communication between the filtrate portion 308 of the filter 304 and a second filter outlet line 324. The example second filter outlet line 324 provides fluid communication between the second outlet 322 and the recirculation circuit 302. The example second filter outlet line 324 includes a second valve 326 which controls flow of fluid from the second outlet 322 into the recirculation circuit 302.

The example filter 304 of FIG. 24A further includes a third outlet 328 connected to a second end of the concentrate portion 306 of the filter 304. The example third outlet 328 provides fluid communication between the concentrate portion 306 of the filter 304 and a drain line 330. The example drain line 330 provides fluid communication between the third outlet 328 and a drain 332. The example drain line 330 includes a third valve 334 which controls flow of fluid from the third outlet 328 into the drain 332.

The recirculation circuit 302 of FIG. 24A includes a fluid pump 336. The example fluid pump 336 is configured to, via control by a control unit 338, push fluid through the system 300. In the example of FIG. 24A, the fluid pump 336 is illustrated to push fluid into the inlet 312 of the filter 304. In other examples, the fluid pump 336 may push fluid in another direction.

The example system 300 of FIG. 24A further includes a fluid source 340. The example fluid source may include PD solution, PD fluid, or any other sterile fluid suitable for the recirculation and flush process of FIGS. 24A-24B. The example fluid source 340 is connected to the filter supply line 314 via a fluid supply line 342. The example fluid supply line 342 includes a fourth valve 344 which controls fluid of fluid from the fluid source 340 into the filter supply line 314.

In the example of FIG. 24A, the system 300 is configured for recirculation. In the illustrated recirculation configuration, the first valve 320 is open, the second valve 326, the third valve 334, and the fourth valve 344 are closed, and the fluid pump 336 operates to circulate fluid through the recirculation circuit 302. During recirculation, the fluid pump 336 pumps a fixed volume of fluid from the inlet 313 of the filter 304 into the concentrate portion of the filter 304. The fixed volume of fluid may be introduced from the fluid source 340 prior to recirculation. The fluid is then pumped through the membrane 310 into the filtrate portion 308 of the filter 304. The fluid is pumped out of the filter 304 via the first outlet 316 and into the first filter outlet line 318. The fluid continues to be pumped through the recirculation circuit 302 until it again reaches the inlet 312 of the filter 304 and begins to recirculate (e.g., travel the aforementioned fluid flow path an additional time).

In other examples, during recirculation, the first valve 320 may be closed and the second valve 326 may be open. In this example, the fixed volume of fluid is pumped from the filtrate portion 308 of the filter 304, is pumped out of the filter 304 through the second outlet 322, and is pumped through the second filter outlet line 324 into the recirculation circuit 302. In other examples, both the first valve 320 and the second valve 326 may be open, allowing fluid to be pumped out of the filtrate portion 308 of the filter 304 at both the first outlet 316 and the second outlet 322. In other examples, the control unit 338 may instruct the system 300 to alternate opening and closing of the first valve 320 and the second valve 326. For example, for a first period of time, the first valve 320 may be open while the second valve 326 is closed. Then, for a second period of time, the first valve 320 may be closed while the second valve 326 is open. The pattern of the first period of time and the second period of time can be repeated in order to alternate the opening and closing of the first valve 320 and the second valve 326 during the recirculation process.

During recirculation, each time the volume of fluid passes through the membrane, a concentration of target molecules is reduced in the filtrate portion 308 of the filter 304. As such, recirculation of the fluid through the recirculation circuit 302 including the filter 304 works to increase the amount of target molecules excluded (e.g., removed) from the recirculation circuit 302 while only using a set volume of fluid.

FIG. 24B is a schematic of one embodiment of the medical fluid system 300 of the present disclosure showing a flush configuration. The example system 300 may be used to flush the fixed volume of fluid which has been recirculated through the recirculation circuit 302. For example, after the fixed volume of fluid has been recirculated through the recirculation circuit 302, the system 300 may be placed in the flush configuration of FIG. 24B to remove the fixed volume of fluid from the system 300.

In the flush configuration illustrated in FIG. 24B, the first valve 320 and the second valve 326 are closed and the third valve 334 and the fourth valve 344 are open. Because the fourth valve 344 is open, the fluid pump 366 can pull a volume of fresh fluid from the fluid source 340 into the fluid supply line 342 and push the volume of fresh fluid through the filter supply line 314 into the inlet 312 of the filter 304. The volume of fresh fluid is pumped into the concentrate portion 306 of the filter 304 and is pumped out through the third outlet 328 of the filter. While traveling through the concentrate portion 306 of the filter 304, the volume of fresh fluid combines with the target molecules that accumulated in the concentrate portion 306 during the recirculation process. As such, when the volume of fresh fluid exits the concentrate portion 306 of the filter 304, the volume of fresh fluid removes at least a portion of the target molecules from the filter 304.

The volume of fresh fluid combined with the portion of the target molecules travels down the drain line 330 into the drain 332, thus removing the volume of fresh fluid and the portion of the target molecules from the system 300. Using the recirculation and flush processes disclosed in conjunction with FIGS. 24A-24B, a high logarithmic reduction value (LRV) of target molecules from the system 300 can be achieved using only a minimal, fixed volume of fluid.

In some examples, an increased LRV can be achieved by repeating the process of FIGS. 24A and 24B a number of times. For example, a first volume of fluid may be introduced and recirculated through the recirculation circuit 302 and then flushed using a first flush volume of fluid. Subsequently, a second volume of fluid may be introduced, recirculated and flushed using a second flush volume of fluid. Repeating the recirculation and flush steps of FIGS. 24A and 24B may increase an LRV of target molecules while still using a reduced amount of fluid compared to traditional flush methods.

### Multiple Ultrafilter Configuration Embodiment

FIG. 25 is a schematic of one embodiment of the medical fluid system 400 of the present disclosure showing a multiple ultrafilter configuration. The example medical fluid system 400 of FIG. 25 may be substantially similar to the system 10 of FIG. 1 except for the changes disclosed below. For example, the example medical fluid system 400 may not include the disposable filter set 40. Additionally or alternatively, the medical fluid system 400 employs a set of two or more ultrafilters. In some examples. to improve usability, the two or more ultrafilters in an ultrafilter box 402 as illustrated in FIG. 25. The example ultrafilter box 402 is illustrated as enclosed within a housing 22 of the system 400. The example ultrafilter box 402 includes a box housing 404 enclosing a first ultrafilter 406 and a second ultrafilter 408. The example ultrafilter box 402 further includes an inlet 410 and an outlet 412.

The example first ultrafilter 406 includes a first housing 414 enclosing a first chamber 416 and a second chamber 418 separated by a first membrane 420. The example first chamber 416 may represent a concentrate side of the first ultrafilter 406 and the second chamber 418 may represent a the filtrate side of the first ultrafilter 406. While the example first ultrafilter 406 is illustrated as having a single, flat membrane, it should be understood that the first membrane 420 may be comprised of a plurality of membranes (e.g., hollow fibers) separating the first chamber 416 (e.g., the concentrate side) and the second chamber 418 (e.g., the filtrate side).

The example first ultrafilter 406 further includes a first outlet 422 which provides communication between the first chamber 416 (e.g., the concentrate side) of the first ultrafilter 406 and a drain or recirculation line (not illustrated) of the medical fluid system 400 in order to remove fluid from the first chamber 416 of the first ultrafilter 406. The inlet 410 of the ultrafilter box 402 is connected to the first chamber 416 of the first ultrafilter 406.

The example second ultrafilter 408 includes a second housing 426 enclosing a first chamber 428 and a second chamber 430 separated by a second membrane 432. The example first chamber 428 may represent a concentrate side of the second ultrafilter 408 and the second chamber 430 may represent a the filtrate side of the second ultrafilter 408. While the example second ultrafilter 408 is illustrated as having a single, flat membrane, it should be understood that the second membrane 432 may be comprised of a plurality of membranes (e.g., hollow fibers) separating the first chamber 428 (e.g., the concentrate side) and the second chamber 4430 (e.g., the filtrate side).

The example second ultrafilter 408 further includes a second outlet 434 which provides communication between the first chamber 428 (e.g., the concentrate side) of the second ultrafilter 408 and a drain or recirculation line (not illustrated) of the medical fluid system 400 in order to remove fluid from the first chamber 428 of the second ultrafilter 408. The outlet 412 of the ultrafilter box 402 is connected to the second chamber 430 of the second ultrafilter 408. A coupling line 424 provides a connection between the first chamber 428 of the second ultrafilter 408 and the second chamber 418 of the first ultrafilter and the second ultrafilter 408.

The outlet 412 of the example ultrafilter box 402 of FIG. 25 extends outside of the housing 22 of the system 400 and connects to a patient line 436. In the example of FIG. 25, the patient line 436 provides connection to a patient without the use of a disposable filter (e.g., a filter that is disposed of after a single use). The example patient line 436 further connects to a drain line 438 of the example system 400.

In one example, the example ultrafilter box 402 of FIG. 25 is configured to filter fluid before fluid is delivered to a patient. For example, a control unit (not illustrated) may control a fluid pump (not illustrated) of the system 400 to pump a dialysis fluid into the inlet 410 of the ultrafilter box 402. The dialysis fluid is pumped through the first chamber 416 of the first ultrafilter 406, and across the first membrane 420 to the second chamber 418 of the first ultrafilter 406. When the fluid travels across the first membrane 420, contaminants in the fluid are excluded from traveling across the first membrane 420 into the second chamber 418. As such, a concentration of contaminants in the fluid is reduced after the fluid travels across the first membrane 420. Subsequently, the fluid is pumped through the coupling line 424 to the first chamber 428 of the second ultrafilter 408, and across the second membrane 432 to the second chamber 430 of the second ultrafilter 408. When the fluid travels across the second membrane 432, additional contaminants in the fluid are excluded from traveling across the second membrane 432, thus reducing the concentration of contaminants in the fluid further. The filtered fluid then exits the ultrafilter box 402 and the housing 22 via the outlet 412 and is pumped into the patient line 436 for delivery to the patient.

In another example, the example ultrafilter box 402 is configured to filter during testing, cleaning, or maintenance of the system 400. For example, the control unit may control the fluid pump to pump a fluid may into the ultrafilter box 402 via the inlet 410 and travel the path described above for filtration. The fluid may then exit the ultrafilter box 402 at the outlet 412 and travel directly to the drain line 438 without delivery to a patient. To facilitate this configuration, a valve, cap, or clamp (not illustrated) may be placed on the patient line 436 to prevent delivery of the fluid to the patient or down the patient line 436.

In the example of FIG. 25, the patient line 436 is replaced approximately daily for patients using the system 400 for peritoneal dialysis. In contrast, the interval at which the ultrafilter box 402 may need to be replaced in order to maintain adequate filter functioning of the ultrafilter box 402 is substantially longer than one day. For example, the ultrafilter box 402 may be replaced every 1 month, every 2 months, every 3 months, every 6 months, or any other interval substantially longer than one day and suitable for maintaining adequate filter functioning of the ultrafilter box 402. The example ultrafilter box 402 of FIG. 25 provides a user-friendly form for a patient to replace a series of two or more ultrafilters. For example, the ultrafilter box 402 may be produced in a sterile manner at a manufacturing site and delivered to a patient with a home PD system (e.g., the system 400). The patient may open the housing 22 of the system 400, remove a used ultrafilter box, and insert the new ultrafilter box 402.

It should be understood that various changes and modifications to the presently preferred embodiments described herein will be apparent to those skilled in the art. It is therefore intended that such changes and modifications be covered by the appended claims. For example, the ultrafilter box 402 may include more than two ultrafilters. Alternatively, the ultrafilter box 402 may include two or more ultrafilters in a configuration other than the series configuration illustrated in FIG. 25.

In some examples, the two or more ultrafilters are each individually connected to the medical fluid system 400. For example, the ultrafilter box 402 and the coupling line 424 illustrated in FIG. 25 may be omitted. Instead, the first ultrafilter 406 may have a drain line connected to the second chamber 418 which provides fluid connection to a drain or recirculation line of the system 400. Further, the second ultrafilter 408 may have a supply line connected to the first chamber 428 which provides fluid connection to a supply or recirculation line of the system 400. In this manner, each of the two ultrafilters may be individually maintained, replaced, and tested.

### Conclusion

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A peritoneal dialysis (“PD”) system comprising:
a housing;
a PD fluid pump housed by the housing;
a filter including a first outer chamber, a central portion, a first membrane separating the first outer chamber and the central portion, an inlet connected to the first outer chamber, an outlet, a first venting port connected to the first outer chamber, the first venting port having a valve preventing air flow into the filter via the first venting port, and a second venting port connected to the central portion;
a pressure sensor; and
a control unit configured to control the PD fluid pump, the control unit further configured to determine a pressure inside the filter based on an output from the pressure sensor and determine an integrity status of the first membrane based on the pressure inside the filter.

2. The PD system of claim 1, wherein the filter is an ultrafilter, a membrane filter, or a sterile membrane filter.

3. The PD system of claim 1, wherein the filter is an ultrafilter, the first membrane includes a plurality of hollow fibers, the first outer chamber is a concentrate side of the filter, and the central portion is a filtrate side of the filter.

4. The PD system of claim 1, wherein the filter further includes a second outer chamber and a second membrane separating the central portion and the second outer chamber and the control unit is further configured to determine an integrity state of the second membrane based on the pressure inside the filter.

5. The PD system of claim 4, wherein the filter further includes at least one of a first supporting structure between the first outer chamber and the central portion and a second supporting structure between the central portion and the second outer chamber.

6. The PD system of claim 1, wherein the filter further includes a supply line connected to the inlet and a drain line connected to the outlet.

7. The PD system of claim 1, wherein the filter further includes a housing and wherein the first outer chamber and the central portion are within the housing.

8. A peritoneal dialysis (“PD”) system comprising:
a housing;
a PD fluid pump housed by the housing;
a filter set including a first filter having a first outer chamber and a first central chamber, a second filter having a second central chamber and a second outer chamber, a first membrane separating the first outer chamber and the first central chamber, a second membrane separating the second central chamber and the second outer chamber, an inlet connected to the first outer chamber, an outlet connected to the second outer chamber, a first venting port connected to the first outer chamber, the first venting port having a valve preventing air flow into the filter set via the first venting port, a second venting port connected to the first central chamber, and a third venting port connected to the second central chamber;
a pressure sensor; and
a control unit configured to control the PD fluid pump, the control unit further configured to determine a pressure inside the filter set based on an output from the pressure sensor and determine an integrity status of the first membrane and the second membrane based on the pressure inside the filter set.

9. The PD system of claim 8, wherein the first filter is one of an ultrafilter or a membrane filter and the second filter is one of an ultrafilter or a membrane filter.

10. The PD system of claim 8, wherein the filter set further includes a first housing and a second housing.

11. The PD system of claim 10, wherein the first outer chamber and the first central chamber are within the first housing and the second central chamber and the second outer chamber are within the second housing.

12. The PD system of claim 8, further including fluid connection between the first filter and the second filter.

13. A peritoneal dialysis (“PD”) method for testing integrity of a filter, the method comprising:
causing a PD fluid pump, via a control unit of a PD machine, to fill the filter with fluid via an inlet of the filter;
causing the PD fluid pump, via the control unit, to remove a portion of the fluid via a drain line until a pressure in the filter reaches a first pressure threshold;
causing the PD fluid pump, via the control unit, to refill the filter with fluid via the inlet of the filter until the pressure in the filter is substantially equalized, wherein refilling the filter with fluid causes a portion of a drain line of the filter proximate to a cap to fill with fluid;
causing the PD fluid pump, via the control unit, to remove a second portion of the fluid via the inlet of the filter until a pressure in a first chamber of the filter reaches a second pressure threshold;
monitoring, via a pressure sensor controlled by the control unit, the pressure in the first chamber for a first period of time; and
determining, via the control unit, an integrity status of a first membrane of the filter based on the pressure in the first chamber during the first period of time.

14. The PD method of claim 13, further comprising causing the filter, via the control unit, to equalize the pressure in the first chamber after monitoring the pressure in the first chamber.

15. The PD method of claim 13, further comprising determining, via the control unit, the first membrane does not include at least one hole or deformity in response to the pressure in the first chamber remaining stable for the first period of time.

16. The PD method of claim 13, further comprising determining, via the control unit, the first membrane includes one or more holes or deformities in response to the pressure in the first chamber increasing or decreasing during the first period of time.

17. The PD method of claim 13, further comprising:
causing the PD fluid pump, via the control unit, to remove a third portion of the fluid via the drain line until a pressure in a second chamber of the filter and a pressure in the first chamber of the filter reach a third pressure threshold;
monitoring, via the pressure sensor controlled by the control unit, the pressure in the first chamber and the second chamber for a second period of time; and
determining, via the control unit, an integrity status of a second membrane of the filter based on the pressure in the first chamber and the second chamber during the second period of time.

18. The PD method of claim 17, further comprising communicating, via the control unit, the integrity status of the first membrane and the integrity status of the second membrane.

19. The PD method of claim 17, further comprising determining, via the control unit, the second membrane does not include a at least one hole or deformity in response to the pressure in the first chamber and the second chamber remaining stable for the second period of time.

20. The PD method of claim 17, further comprising determining, via the control unit, the second membrane includes one or more holes or deformities in response to the pressure in the first chamber and the second chamber increasing or decreasing during the second period of time.

21. A peritoneal dialysis (“PD”) method for removing contaminants, the method comprising:
opening, via a control unit of a PD machine, a first valve connected to a first outlet of a filter;
closing, via the control unit, a second valve connected to a second outlet of the filter and a third valve connected to a third outlet of a filter;
controlling, via the control unit, a pump to push a first volume of fluid from a concentrate side to a filtrate side of the filter, wherein the fluid flows from an inlet of the filter to the first outlet of the filter, passing through one or more membranes of the filter, wherein the fluid flows through a recirculation circuit back to the inlet of the filter after flowing out the first outlet of the filter;
closing, via the control unit, the first valve of the filter;
opening, via the control unit, the third valve of the filter and a fourth valve, the fourth valve providing fluid connection between a fluid source and the recirculation circuit; and
controlling, via the control unit, the pump to push a second volume of fluid from the fluid source and the first volume of fluid to a drain, wherein the first volume and the second volume flow from the inlet of the filter to the third outlet of the filter before flowing to the drain.

22. The PD method of claim 21, further including, prior to the opening of the third valve and the fourth valve,
closing, via the control unit, the first valve of the filter; and
opening, via the control unit, the second valve of the filter, wherein the fluid flows from the inlet of the filter to the second outlet of the filter before flowing through the recirculation circuit.

23. The PD method of claim 21, further including:
closing, via the control unit, the third valve and the fourth valve;
opening, via the control unit, the first valve; and
controlling, via the control unit, the pump to push a third volume of fluid through the recirculation circuit.
